(19) United States
(12) Patent Application Publication
(10) Pub. No.: US 2019/0329199 A1
(43) Pub. Date: Oct. 31, 2019

(54) FINE BUBBLE GENERATION PROMOTER, FINE-BUBBLE-CONTAINING LIQUID, AND METHOD AND DEVICE FOR PRODUCING FINE-BUBBLE-CONTAINING LIQUID

(71) Applicant: PANASONIC INTELLECTUAL PROPERTY MANAGEMENT CO., LTD, Osaka (JP)

(72) Inventors: Kou SUGANO, Osaka (JP); Yuichi MIYOSHI, Osaka (JP); Shigeru TSUZUKI, Osaka (JP); Sachiko INAZATO, Osaka (JP)

(21) Appl. No.: 16/462,160

(22) PCT Filed: Nov. 16, 2017
(86) PCT No.: PCT/JP2017/041184

§ 371 Date: May 17, 2019

(30) Foreign Application Priority Data
Nov. 24, 2016 (JP) 2016-227702
Dec. 26, 2016 (JP) 2016-250335

(51) Int. Cl. B01F 17/00; B01F 3/04; B01F 5/02; A23L 29/10; C12G 3/04; B08B 3/08; A61K 49/22; A01N 25/24; A61Q 19/00; A61K 8/67; A61K 8/31; A61K 8/36; A61K 8/06; C11D 3/00; C11D 3/20

(52) U.S. Cl. CPC B01F 17/0092; A61L 2/18; B01F 5/02; A23L 29/10; C12G 3/04; B08B 3/08; A61K 49/223; A01N 25/24; A61Q 19/00; A61K 8/67; A61K 8/31; A61K 8/361; A61K 8/068; B01F 17/0021; C11D 3/0094; C11D 3/2079; C11D 3/2096; B01F 2215/0014; B01F 2215/0031; B01F 2215/0034; B01F 2215/007; B01F 2215/0077; A23V 2002/00; B01F 3/0446

(57) ABSTRACT

A fine bubble generation promoter includes: one of a fatty acid and a fat-soluble vitamin in an amount within a range of 2.4 wt % to 33 wt %, inclusive; and a hydrocarbon in an amount within a range of 67 wt % to 97 wt %, inclusive. A total concentration of (i) one of the fatty acid and the fat-soluble vitamin and (ii) the hydrocarbon is 99 wt % or more.

### TECHNICAL FIELD

The present invention relates to a fine bubble generation promoter, a fine-bubble-containing liquid, and a method and device for producing a fine-bubble-containing liquid.

### BACKGROUND ART

In recent years, the practical application of liquids containing minute bubbles has been proceeding in a large number of fields such as for promoting growth of fish and plants, efficient treatment of industrial waste, removing an oil content of industrial products, medical treatment applications, cosmetics applications, and also for applications in the food sector. Although the bubbles that have conventionally been utilized as these minute bubbles are bubbles each having a size ϕ in the range of approximately 1 μm to 300 μm that are referred to as “microbubbles”, in recent years the usefulness of fine-bubble-containing liquids that contain nano-order fine bubbles having a mean size of 0.8 μm or less has been demonstrated in various industrial uses.

As a common method for generating fine bubbles, a method may be mentioned in which a gaseous matter is sucked into a liquid and is pressurized and dissolved until a supersaturated state is reached, and a high pressure is then applied to the liquid using a Venturi tube, a swirl flow nozzle that has a rotor, or a porous body having micro-order pores.

In PTL 1, a method for generating fine bubbles is disclosed that uses a pressurized dissolution method in which a high pressure is applied to a liquid containing two kinds of surface-active agents.

Further, in PTL 2, a method for generating fine bubbles is disclosed in which a surface-active agent and gaseous matter are supplied to a generating device, and the resultant solution is pressurized in a porous body having a mean pore diameter within a range of 2 μm to 30 μm to thereby generate fine bubbles.

### CITATION LIST

### Patent Literature

PTL 1: Japanese Unexamined Patent Application Publication No. 2007-314463

PTL 2: Japanese Unexamined Patent Application Publication No. 2016-123925

### SUMMARY OF THE INVENTION

### Technical Problem

However, conventionally, it has been difficult to maintain a high concentration of fine bubbles for an extended period of time in such kind of liquid containing nano-order fine bubbles.

Therefore, an object of the present invention is to provide a fine bubble generation promoter, a fine-bubble-containing liquid, and a method and device for producing a fine-bubble-containing liquid which can maintain a high concentration of fine bubbles for an extended period of time.

### Solutions to Problem

In order to achieve the above-described object, in accordance with an aspect of the present invention, there is provided a fine bubble generation promoter, including: one of a fatty acid and a fat-soluble vitamin; and hydrocarbon, wherein a weight ratio between (i) the one of the fatty acid and the fat-soluble vitamin and (ii) the hydrocarbon is within a range of 1:2 to 1:40, inclusive.

In order to achieve the above-described object, in accordance with another aspect of the present invention, there is provided a fine bubble generation promoter, including: one of a fatty acid and a fat-soluble vitamin, the one of the fatty acid and the fat-soluble vitamin being in an amount within a range of 2.4 wt % to 33 wt %, inclusive; and a hydrocarbon in an amount within a range of 67 wt % to 97 wt %, inclusive, wherein a total concentration of (i) the one of the fatty acid and the fat-soluble vitamin and (ii) the hydrocarbon is 99 wt % or more.

In order to achieve the above-described object, in accordance with still another aspect of the present invention, there is provided a fine-bubble-containing liquid, including: water; one of a fatty acid and a fat-soluble vitamin, the fatty acid comprising only carbon, oxygen, and hydrogen; and fine bubbles, wherein the fine bubbles each have a size within a range of 1 nm to 800 nm, inclusive.

In order to achieve the above-described object, in accordance with still another aspect of the present invention, there is provided a fine-bubble-containing liquid, including: water; the fine bubble generation promoter according to claim 1 or 2; and fine bubbles, wherein the fine bubbles each have a size within a range of 1 nm to 800 nm, inclusive.

In accordance with still another aspect of the present invention, there is provided a method for producing a fine-bubble-containing liquid, the method including: generating a fine bubble generation promoting liquid by adding one of a fatty acid and a fat-soluble vitamin to water, the fatty acid comprising only carbon, oxygen, and hydrogen; and generating fine bubbles each having a size within a range of 1 nm to 800 nm, inclusive, in the fine bubble generation promoting liquid, without introducing a gaseous matter from outside.

In accordance with still another aspect of the present invention, there is provided a device for producing a fine-bubble-containing liquid, the device including: a water supply valve that supplies water; piping that is a channel in which the water flows; a pump that pumps the water; an injection valve that injects one of a fatty acid or a fat-soluble vitamin into the water, the fatty acid comprising only carbon, oxygen, and hydrogen; and a fine bubble generator that generates a fine-bubble-containing liquid having fine bubbles from the water and a fine bubble generation promoting liquid comprising the one of the fatty acid and the fat-soluble vitamin, wherein the device for producing a fine-bubble-containing liquid does not comprise an introduction valve for introducing a gaseous matter into the water from outside.

### Advantageous Effects of Invention

According to the present invention, a fine bubble generation promoter, a fine-bubble-containing liquid, and a method and device for producing a fine-bubble-containing liquid which can maintain a high concentration of fine bubbles for an extended period of time can be provided.

### BRIEF DESCRIPTION OF DRAWINGS

FIG. 1 is a view illustrating a relation between a bubble size and a bubble concentration of a fine bubble liquid of a sample A.

FIG. 2 is a view illustrating a relation between a bubble size and a bubble concentration of a fine bubble liquid of a sample B.

FIG. 3 is a view illustrating a relation between a bubble size and a bubble concentration of a fine bubble liquid of a sample C.

FIG. 4 is a view illustrating a relation between a bubble size and a bubble concentration of a fine bubble liquid of a sample D.

FIG. 5 is a view illustrating a relation between a bubble size and a bubble concentration of a fine bubble liquid of a sample E.

FIG. 6 is a view illustrating a relation between a concentration of an additive agent and a concentration of fine bubbles according to Embodiment 1.

FIG. 7 is a view showing a TEM image of fine bubble according to Embodiment 1.

FIG. 8 is a view showing the TEM image of the fine bubble according to Embodiment 1.

FIG. 9 is a view showing the TEM image of the fine bubble according to Embodiment 1.

FIG. 10 is a configuration diagram of a device for producing a fine-bubble-containing liquid according to Embodiment 1.

FIG. 11 is a view illustrating a relation between a carbon number and a concentration of fine bubbles according to Embodiment 2.

FIG. 12 is a view showing examples of fatty acids and a fat-soluble vitamin according to Embodiment 2.

FIG. 13 is a view illustrating a relation between a concentration of an additive agent and a concentration of fine bubbles according to Embodiment 2.

FIG. 14 is a view illustrating a relation between an electric resistivity and a concentration of the fine bubbles according to Embodiment 2.

FIG. 15A is a view illustrating a relation between zeta potential and a concentration of the fatty acid according to Embodiment 2.

FIG. 15B is a view illustrating a relation between zeta potential and a concentration of surface-active agents according to a Conventional Example.

FIG. 16 is a view showing TEM images of the fine bubbles which the additive agents adsorbed according to Embodiment 2.

FIG. 17A is a view illustrating distribution of sizes of the fine bubbles in a case where oleic acid is added according to Embodiment 2.

FIG. 17B is a view illustrating the distribution of the sizes of the fine bubbles in a case where α-tocopherol was added according to Embodiment 2.

FIG. 18 is a view illustrating a lifetime of the fine bubbles according to Embodiment 2.

FIG. 19 is a flowchart illustrating a flow of a method for generating a fine-bubble-containing liquid according to Embodiment 2.

### DESCRIPTION OF EXEMPLARY EMBODIMENTS

Hereinafter, certain exemplary embodiments are described in greater detail with reference to the accompanying Drawings. However, unnecessarily detailed description may be omitted. For example, detailed explanation of a well-known matter and repeated description of substantially identical structures may be omitted. Such omission makes the following description exclude unnecessary redundancy and be easily understood by those skilled in the art. Furthermore, each figure in the Drawings is a schematic diagram and is not necessarily an exact diagram.

(Regarding Size, Concentration and Method for Measurement of Fine Bubbles)

In recent years it has been understood that liquids containing nano-order fine bubbles (hereinafter, also referred to as “ultrafine bubbles”) have useful properties with respect to various industrial uses. In order for such liquids containing nano-order fine bubbles to sufficiently exhibit those properties, it is preferable that the liquid contains a concentration of 1×10⁹ per ml or more of bubbles each having a size within a range of 1 nm to 0.8 μm (800 nm), and for practical use it is preferable that a lifetime of a concentration of such bubbles is 30 days or more.

Hereunder, the term “fine bubbles” (or “nano-order fine bubbles”) as used in the respective embodiments means bubbles each having a size within a range of 1 nm to 0.8 μm, and the targeted concentration of the fine bubbles (hereunder, referred to as a “target concentration”) is a concentration of 1×10⁹ per ml or more, and the targeted lifetime (hereunder, referred to as a “target lifetime”) is for the target concentration to be maintained for 30 days or more.

In the respective embodiments described hereunder, measurement of the size (for example, diameter) and the concentration of the fine bubbles was performed using a nano-tracking method to track fine bubbles that performed a Brownian motion that was confirmed with scattered laser light, and the size and concentration (percentage content) were calculated rheologically using the Stokes-Einstein formula. An LM10 instrument manufactured by NanoSight Ltd was used as the device for measuring the size and concentration (percentage content) of the fine bubbles, and measurement was performed at 25° C. and 1 atm.

### Embodiment 1

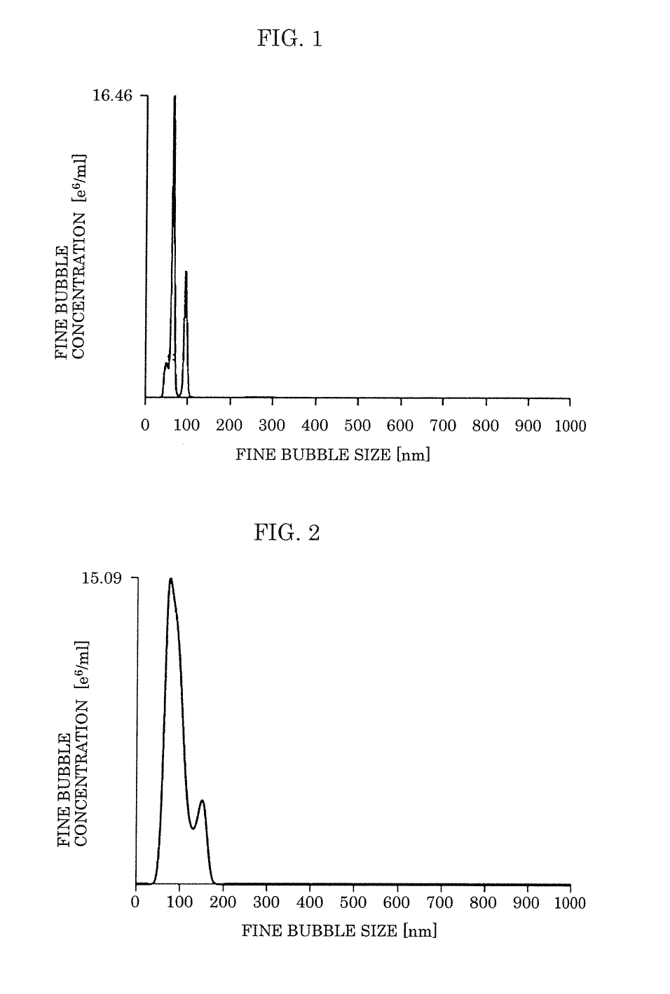

The present embodiment will be described with reference to FIG. 1 to FIG. 10.

[1-1. Fine-Bubble-Containing Liquid]

For each of samples A to E that are described hereunder, fine bubbles were generated by causing various adjusted liquids to jet out from a nozzle in a hermetically sealed flow channel, and a concentration and a lifetime of the fine bubbles were measured. The details of a method for generating the fine bubbles are described later in the section regarding a method for producing a fine-bubble-containing liquid.

Sample A: Only ultrapure water with an electric resistivity of 18 MΩ·cm was used as the liquid, and an additive agent or the like was not contained in the liquid.

FIG. 1 is a view illustrating a relation between a size and a concentration of fine bubbles in a fine-bubble-containing liquid of sample A. As illustrated in FIG. 1, the concentration of the generated fine bubbles was approximately 2×10⁸ per ml, and hence the target concentration of fine bubbles could not be obtained.

Sample B: A liquid was used that was obtained by using ultrapure water with an electric resistivity of 18 MΩ·cm as a solvent, and adding 20 ppm of oleic acid as an additive agent to the solvent.

FIG. 2 is a view illustrating a relation between a size and a concentration of fine bubbles in a fine-bubble-containing liquid of sample B. As illustrated in FIG. 2, the concentration of the generated fine bubbles was approximately 8×10⁸ per ml, and hence the target concentration of fine bubbles could not be obtained.

Sample C: A liquid was used that was obtained by using ultrapure water with an electric resistivity of 18 MΩ·cm as a solvent, and adding 400 ppm of heptane as an additive agent to the solvent.

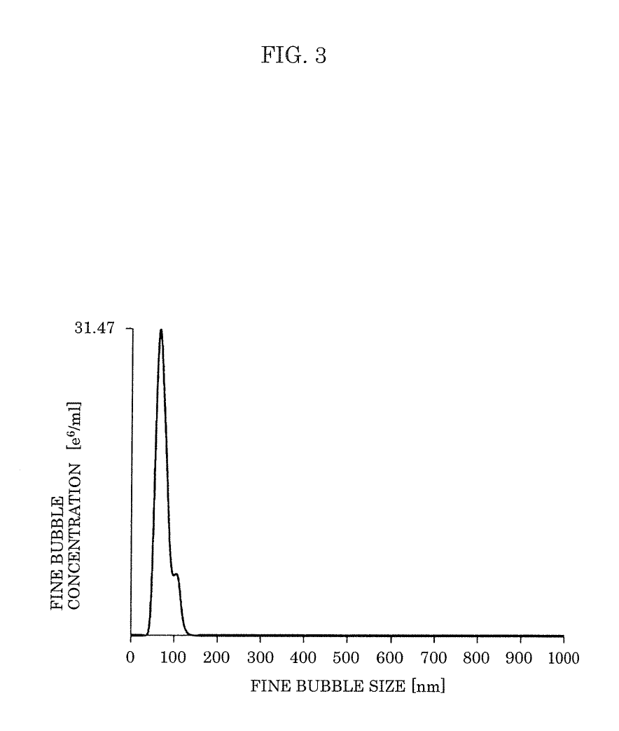

FIG. 3 is a view illustrating a relation between a size and a concentration of fine bubbles in a fine-bubble-containing liquid of sample C. As illustrated in FIG. 3, the concentration of the generated fine bubbles was approximately 6.8×10⁸ per ml, and hence the target concentration of fine bubbles could not be obtained.

Sample D: A liquid was used that was obtained by using ultrapure water with an electric resistivity of 18 MΩ·cm as a solvent, and adding 20 ppm of oleic acid and 400 ppm of heptane as additive agents to the solvent.

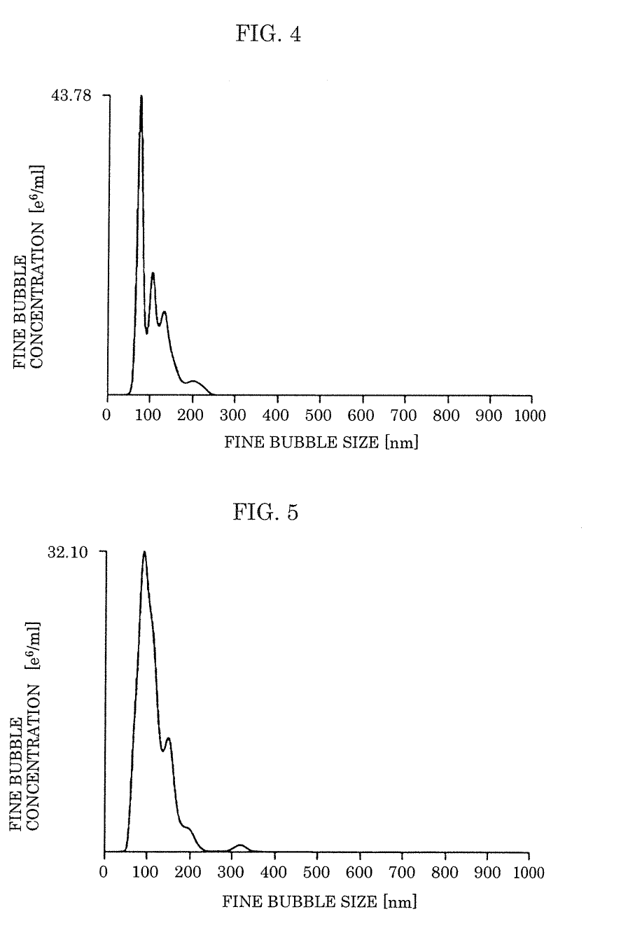

FIG. 4 is a view illustrating a relation between a size and a concentration of fine bubbles that were measured when the fine-bubble-containing liquid of sample D was diluted 20-fold. As illustrated in FIG. 4, the concentration of fine bubbles generated using this liquid was approximately 4.5×10⁹ per ml, and thus fine bubbles of an amount equal to or greater than the target concentration could be obtained. Further, the concentration of fine bubbles that was equal to or greater than the target concentration could be maintained for 30 days or more after generation of the fine bubbles. At this time, the mean size ϕ of the fine bubbles was approximately 100 nm (90 to 110 nm), and the peak size ϕ was approximately 75 nm. An electric resistivity of the liquid of sample C was approximately 2 to 5 MΩ·cm, and thus an electric resistivity of 1 MΩ·cm or more could be secured.

Sample E: A liquid was used that was obtained by using ultrapure water with an electric resistivity of 18 MΩ·cm as a solvent, and adding 50 ppm of oleic acid and 200 ppm of heptane as additive agents to the solvent.

FIG. 5 is a view illustrating a relation between a size and a concentration of fine bubbles that were measured when the fine-bubble-containing liquid of sample E was diluted 10-fold. As illustrated in FIG. 5, the concentration of fine bubbles generated using this liquid was approximately 2.1×10¹⁰ per ml, and thus fine bubbles of concentration equal to or greater than the target concentration could be obtained. Further, the concentration of fine bubbles that was equal to or greater than the target concentration could be maintained for 30 days or more after generation of the fine bubbles. At this time, the mean size ϕ of the fine bubbles was approximately 110 nm (100 to 120 nm), and the peak size ϕ was approximately 90 nm. An electric resistivity of the liquid of sample E was around 2 to 5 MΩ·cm, and thus an electric resistivity of 1 MΩ·cm or more could be secured.

In addition, liquids were prepared in which ultrapure water with an electric resistivity of 18 MΩ·cm was used as a solvent, and oleic acid whose concentration was varied between 10 ppm, 20 ppm, and 50 ppm and heptane whose concentration was varied between 100 ppm, 200 ppm, and 400 ppm were added to the solvent as additive agents, and the concentration of fine bubbles in fine-bubble-containing liquids that were prepared using these respective liquids were measured. That is, the concentrations of fine bubbles in a plurality of kinds of fine-bubble-containing liquids prepared by adding oleic acid and heptane as additive agents to ultrapure water in a manner in which the concentrations of the oleic acid and heptane were varied were measured.

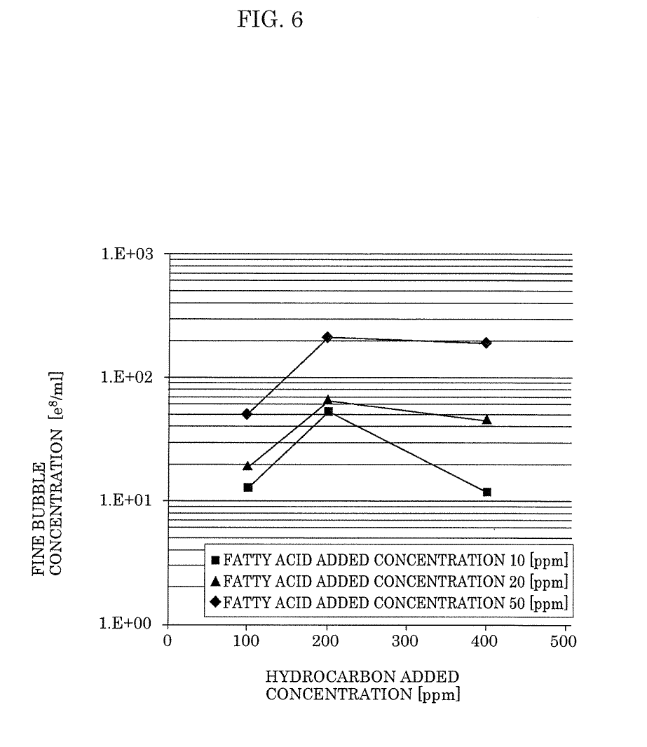

FIG. 6 is a view illustrating a relation between a concentration of additive agents and a concentration of fine bubbles. As illustrated in FIG. 6, within a range in which the concentration of oleic acid was from 10 to 50 ppm and the concentration of heptane was from 100 to 400 ppm, the fine-bubble-containing liquids could generate fine bubbles with a concentration equal to or greater than 1×10⁹ per ml which is the target concentration. Further, the electric resistivity of the fine-bubble-containing liquids according to the respective conditions described above was 1 MΩ·cm or more. In addition, even after a period of 30 days or more elapsed, the fine-bubble-containing liquids according to the respective conditions described above could maintain the concentration of fine bubbles that was equal to or greater than the target concentration. Furthermore, as the result of continuing to check the concentration of fine bubbles it was confirmed that a concentration of fine bubbles that was equal to or greater than the target concentration was maintained even after a period of 18 months passed.

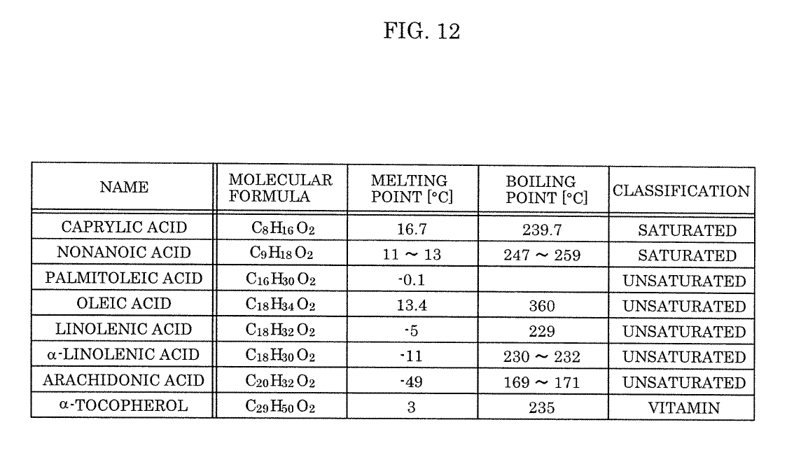

In addition, instead of the oleic acid that is used as the additive agent in the fine-bubble-containing liquid according to the present embodiment, the fine-bubble-containing liquid can be produced that similarly contains a high concentration of fine bubbles with a long lifetime by using another fatty acid such as caprylic acid, nonanoic acid, palmitoleic acid, linoleic acid, α-linolenic acid, or arachidonic acid, or a fat-soluble vitamin (see FIG. 12). In this case, fatty acids that can be used are saturated fatty acids having a carbon number from 5 to 12 or unsaturated fatty acids having a carbon number of 12 or more, and, for example, α-tocopherol can be used as a fat-soluble vitamin. Because these fatty acids or fat-soluble vitamins can be adsorbed by fine bubbles and can stably disperse without aggregating in the liquid, it is possible to generate a high concentration of fine bubbles and it is also possible to prolong the lifetime of the fine bubbles. Note that the fatty acids are composed only of carbon, oxygen, and hydrogen.

Further, a fine-bubble-containing liquid can be produced that similarly contains a high concentration of fine bubbles with a long lifetime by using another hydrocarbon such as hexane, octane, nonane, or decane as the additive agent instead of heptane in the fine-bubble-containing liquid according to the present embodiment. The hydrocarbon used in this case is preferably an alkane having a carbon number from 5 to 13. By the hydrocarbon having a carbon number from 5 to 13, it is possible to sufficiently increase a critical micelle concentration, and thus favorable surface chemistry properties are obtained, and it is possible to generate the high concentration of fine bubbles even if the concentration of fatty acids or fat-soluble vitamins is quite low.

Furthermore, the hydrocarbon that is used as the additive agent is more preferably a hydrocarbon having a boiling point of 60° C. or higher at 1 atm pressure, and more preferably has a carbon number of not more than 10. When the boiling point of the hydrocarbon is 60° C. or higher at 1 atm pressure, the volatility is low and the stability of the content in the liquid is maintained over an extended period of time, and when the carbon number of the hydrocarbon is not more than 10, more favorable surface chemistry properties can be obtained.

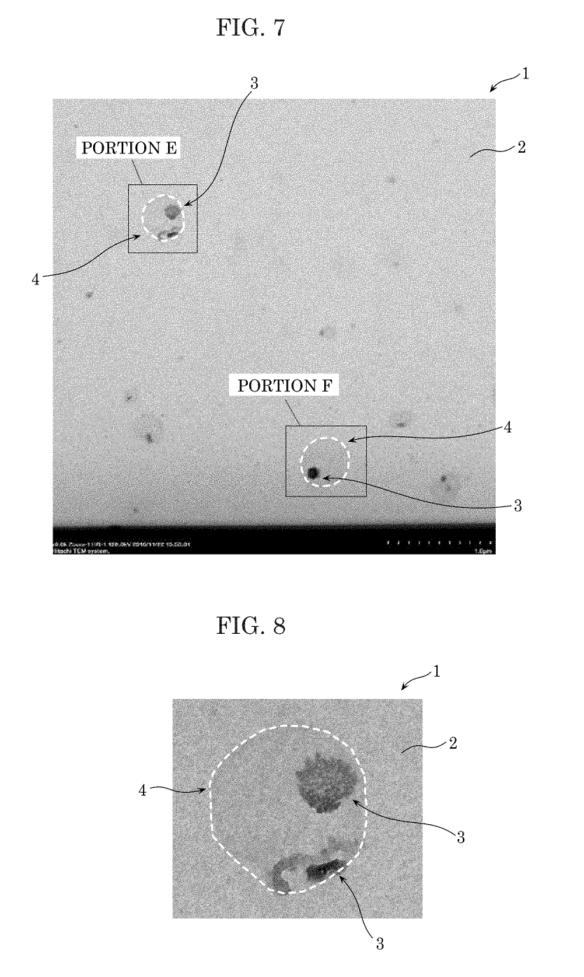

Next, the structure of the fine bubbles will be described with reference to FIG. 7 to FIG. 9.

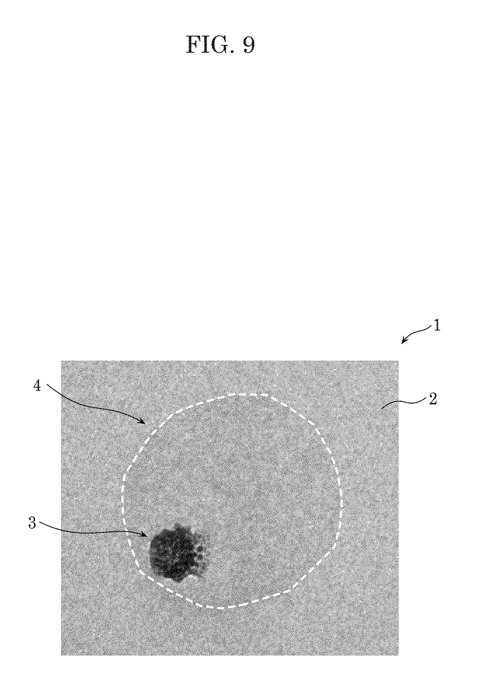

FIG. 7 is a view showing a TEM (transmission electron microscope) image of fine bubbles of sample D according to the present embodiment. FIG. 8 is an enlarged view showing the TEM image of a fine bubble at portion E in FIG. 7. FIG. 9 is an enlarged view showing the TEM image of a fine bubble at portion F in FIG. 7.

In FIG. 7 to FIG. 9, in fine-bubble-containing liquid 1 according to the present embodiment, fine bubbles 4 adsorbed on additive agent 3 are dispersed in liquid 2. In FIG. 7 to FIG. 9, a white dashed line indicates a contour of respective fine bubbles 4. The size of fine bubble 4 shown in portion E is approximately 410 nm, and the size of fine bubble 4 shown in portion F is approximately 450 nm. In FIG. 7 to FIG. 9, additive agent 3 is either one of, or both of, oleic acid and heptane. Adsorption of fine bubbles 4 by additive agent 3 allows fine bubbles 4 to be stabilized and dispersed in a high concentration in liquid 2, and to be present in liquid 2 for an extended period of time.

As described above, the fine-bubble-containing liquid of the present embodiment includes a liquid, and fine bubbles that are dispersed in the liquid. The liquid contains water, a fatty acid or a fat-soluble vitamin, and a hydrocarbon. The size of the fine bubbles is within a range of 1 nm to 0.8 μm. The concentration of the fine bubbles (percentage content) is 1×10⁹ per ml or more.

By having the above-described composition, it is easy to generate fine bubbles having a size within a range of 1 nm to 0.8 μm at a concentration of 1×10⁹ per ml or more in the fine-bubble-containing liquid, and that concentration of fine bubbles can be maintained for 30 days or more. Further, because the concentration of fine bubbles is a high concentration of 1×10⁹ per ml or more, the fine-bubble-containing liquid has a higher cleaning capacity.

Furthermore, it is preferable that a concentration (percentage content) of the one of the fatty acid and the fat-soluble vitamin is within a range of 10 ppm to 50 ppm, inclusive, and that a concentration (percentage content) of the hydrocarbon is within a range of 100 ppm to 400 ppm, inclusive.

By having the above-described composition, it is easy to generate fine bubbles having a size within a range of 1 nm to 0.8 μm at a concentration of 1×10⁹ per ml or more in the fine-bubble-containing liquid, and it is easy to maintain that concentration of fine bubbles for 30 days or more.

In addition, in the fine-bubble-containing liquid, the fatty acid is preferably a saturated fatty acid having a carbon number from 5 to 12 or an unsaturated fatty acid having a carbon number of 12 or more, and the hydrocarbon is preferably an alkane having a carbon number from 5 to 13.

By making the fatty acid contained in the fine-bubble-containing liquid a saturated fatty acid having a carbon number from 5 to 12 or an unsaturated fatty acid having a carbon number of 12 or more, and making the hydrocarbon contained in the fine-bubble-containing liquid an alkane having a carbon number from 5 to 13, it is easy to generate fine bubbles having a size within a range of 1 nm to 0.8 μm at a concentration of 1×10⁹ per ml or more, and maintaining that concentration of fine bubbles for 30 days or more is facilitated.

Furthermore, it is preferable that the hydrocarbon is any one of hexane, heptane, octane, nonane, and decane.

By adopting any one of hexane, heptane, octane, nonane, and decane as the hydrocarbon as described above, a fine-bubble-containing liquid can be produced that has a sufficiently high concentration of fine bubbles and a long lifetime.

Furthermore, it is preferable that the fatty acid is one of an oleic acid, a caprylic acid, a nonanoic acid, a palmitoleic acid, a linoleic acid, an α-linolenic acid, and an arachidonic acid, and that the fat-soluble vitamin is α-tocopherol.

By containing the aforementioned fatty acid or fat-soluble vitamin, a fine-bubble-containing liquid can be produced that has a sufficiently high concentration of fine bubbles and a long lifetime. In addition, because the fatty acid or fat-soluble vitamin to be added is a liquid at a room temperature, the fatty acid or fat-soluble vitamin to be added is easily added to water. In other words, fine-bubble-containing liquid 1a can be easily generated.

Furthermore, it is preferable that an electric resistivity (for example, fine-bubble-containing liquid) is 1 MΩ·cm or more.

By having the above-described composition, the content of ions such as metal ions and halogen ions in the fine-bubble-containing liquid is small, and using this fine-bubble-containing liquid as a cleaning fluid enables the production of highly reliable semiconductor devices.

[1-2. Fine Bubble Generation Promoter]

A fine bubble generation promoter is a solution for easily producing a fine-bubble-containing liquid by mixing the fine bubble generation promoter at a predetermined ratio into a solvent such as pure water and generating fine bubbles. Although water such as distilled water or ion-exchanged water can generally be used as a solvent into which to introduce the fine bubble generation promoter, in the case of producing a fine-bubble-containing liquid to be used for cleaning semiconductors, it is preferable to use ultrapure water with an electric resistivity of 18 MΩ·cm or more.

The fine bubble generation promoter according to the present embodiment includes: a fatty acid or fat-soluble vitamin; and a hydrocarbon. Here, a weight ratio between (i) the fatty acid or fat-soluble vitamin and (ii) the hydrocarbon is within a range of 1:2 to 1:40, inclusive.

By having the above-described composition, the fine-bubble-containing liquid of the present embodiment can be easily produced by injecting the fine bubble generation promoter into a solvent. Added amounts of the fatty acid or fat-soluble vitamin and the hydrocarbon are adjusted so that the added amount of the fatty acid or the fat-soluble vitamin is within the range of 10 ppm to 50 ppm and the added amount of the hydrocarbon is within the range of 100 ppm to 400 ppm with respect to the solvent for generating fine bubbles. By adding the hydrocarbon, a high concentration of fine bubbles can be generated using a small added amount of the fatty acid or fat-soluble vitamin. Further, a high concentration of fine bubbles can be maintained for an extended period of time.

Furthermore, it is preferable that the fine bubble generation promoter includes: a fatty acid or fat-soluble vitamin in an amount within a range of 2.4 wt % to 33 wt %, inclusive; and a hydrocarbon in an amount within a range of 67 wt % to 97 wt %, inclusive, and that a total concentration of (i) the fatty acid or fat-soluble vitamin and (ii) the hydrocarbon is 99 wt % or more.

By having the above-described composition, a large amount of fine-bubble-containing liquid can be produced using a small amount of the fine bubble generation promoter, and thus production of a fine-bubble-containing liquid can be facilitated. For example, a fine-bubble-containing liquid in which a high concentration of fine bubbles can be maintained for an extended period of time can be easily produced.

[1-3. Device and Method for Producing a Fine-Bubble-Containing Liquid]

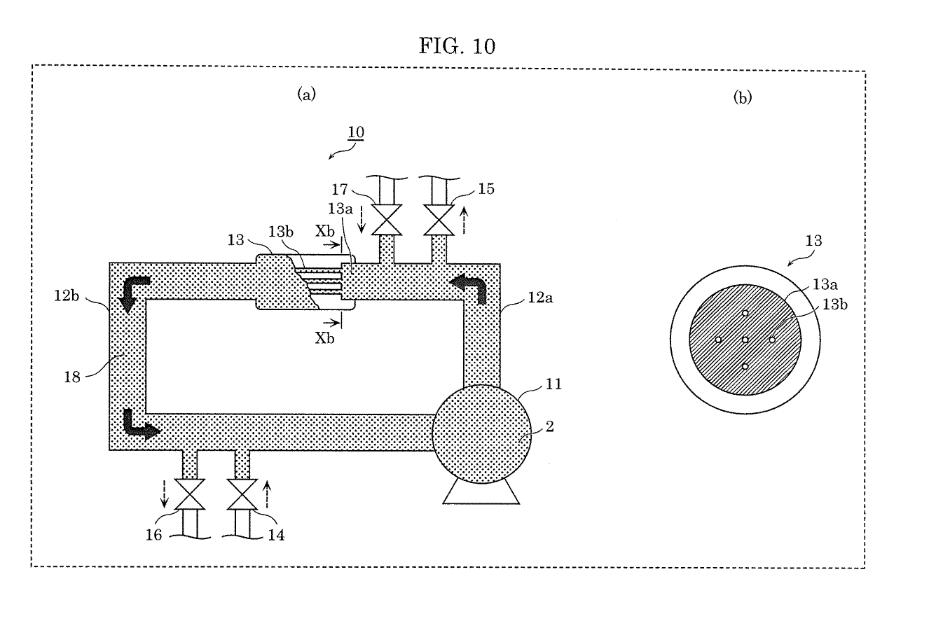

FIG. 10 is a view illustrating device for producing a fine-bubble-containing liquid 10 that is used for producing fine-bubble-containing liquid 1 of the present embodiment. Specifically, (a) in FIG. 10 is a structural diagram of device for producing a fine-bubble-containing liquid 10 according to the present embodiment, and (b) in FIG. 10 is a cross-sectional drawing along line Xb-Xb in (a) in FIG. 10. Note that, (a) in FIG. 10 illustrates a state in which device for producing a fine-bubble-containing liquid 10 is filled with liquid 2 (for example, ultrapure water). Further, the solid-line arrows in (a) in FIG. 10 indicate the direction in which liquid 2 flows inside circulation piping (first piping 12a and second piping 12b). Further, broken-line arrows in (a) in FIG. 10 indicate the directions in which liquid 2, additive agent 3, and the like flow at four valves.

In (a) in FIG. 10, device for producing a fine-bubble-containing liquid 10 includes hermetically sealed flow channel 18 in which circulation piping (for example, first piping 12a and second piping 12b) is connected to pump 11, and nozzle 13 that is provided downstream of pump 11 in hermetically sealed flow channel 18. The circulation piping has four branch pipes. Water supply valve 14 for supplying a solvent, discharge valve 15 for discharging a solution from inside the circulation piping, sampling valve 16 for extracting fine-bubble-containing liquid that is generated, and injection valve 17 for injecting a fine bubble generation promoter are connected to the respective branch pipes.

Pump 11 forms a flow of liquid 2 inside the circulation piping. Pump 11 pumps liquid 2 that flows in from second piping 12b into first piping 12a. In the present embodiment, first piping 12a is connected in the vertically upward direction to pump 11, and pump 11 pumps out liquid 2 in the vertically upward direction.

A bearingless pump in which there are no sliding parts is used as pump 11 according to the present embodiment. By this means, the generation of impurities from pump 11 can be inhibited. In other words, contamination of liquid 2 by impurities from pump 11 can be inhibited. Note that, a pump that can be used as pump 11 is not limited to a bearingless pump.

First piping 12a is connected to pump 11 and nozzle 13, and constitutes a flow channel that is hermetically sealed. Liquid 2 that is pumped out from pump 11 flows inside the hermetically sealed flow channel formed by first piping 12a.

Nozzle 13 generates fine bubbles 4 using fine bubble generation promoting liquid (liquid 2 containing additive agent 3) that flows in from first piping 12a. That is, fine bubbles 4 are generated when the fine bubble generation promoting liquid passes through nozzle 13 (specifically, by the fine bubble generation promoting liquid being jetted out from nozzle pipes 13b) to thereby generate fine-bubble-containing liquid 1. Note that, nozzle 13 is one example of a fine bubble generator.

A plurality of through-holes each having a diameter ϕ within the range of 0.5 ppm to 2.0 mm are formed in the flow direction in each nozzle pipe 13b. The shape of these through-holes may be a forward tapered shape, a reverse tapered shape, or a straight-pipe shape, and the number of the through-holes is preferably within a range of 3 to 20, and more preferably is within a range of 5 to 12.

Second piping 12b is connected to nozzle 13 and pump 11, and constitutes a flow channel that is hermetically sealed. Fine-bubble-containing liquid 1 that is sent out from nozzle 13 flows inside the hermetically sealed flow channel formed by second piping 12b.

Water supply valve 14 is a valve for supplying water to second piping 12b, and is connected to second piping 12b. In the present embodiment, liquid 2 is supplied from water supply valve 14. Note that, water supply valve 14 is directly connected to an ultrapure water generating device (not illustrated), and liquid 2 can be supplied via water supply valve 14 to second piping 12b. By this means, liquid 2 that is generated at the ultrapure water generating device is supplied to device for producing a fine-bubble-containing liquid 10 without being exposed to outside air. In other words, incorporation of impurities contained in outside air into liquid 2 that is caused by exposure to outside air can be inhibited. By this means, it is possible to generate fine-bubble-containing liquid 1 that has a higher electric resistivity.

Discharge valve 15 is connected to first piping 12a, and is a valve for discharging superfluous liquid 2 from inside device for producing a fine-bubble-containing liquid 10. Discharge valve 15 is connected at a location between pump 11 and injection valve 17 of first piping 12a. In the present embodiment, in order to fill pump 11 and the inside of the circulation piping with liquid 2, discharge valve 15 is connected in a manner so that liquid 2 can be taken out in the vertically upward direction from first piping 12a.

Sampling valve 16 is a valve for extracting fine-bubble-containing liquid 1 that is generated, and is connected to second piping 12b. Specifically, sampling valve 16 is connected to second piping 12b at a location between nozzle 13 and water supply valve 14.

Injection valve 17 is connected to first piping 12a at a location between discharge valve 15 and nozzle 13. Injection valve 17 is a valve for supplying additive agent 3 to liquid 2. In the present embodiment, a fatty acid or a fat-soluble vitamin, and a hydrocarbon are supplied into liquid 2 from injection valve 17.

Each constituent element that constitutes device for producing a fine-bubble-containing liquid 10 is formed of a material from which it is difficult for impurities to elute into liquid 2 (difficult for impurities to contaminate ultrapure water). For example, liquid-contacting parts such as pump 11 and the piping are formed of a Teflon (registered trademark) material such as PFA (tetrafluoroethylene-perfluoroalkyl vinyl ether copolymer) or PTFE (polytetrafluoroethylene). By this means, the elution of impurities into liquid 2 from device for producing a fine-bubble-containing liquid 10 can be lowered. In other words, fine-bubble-containing liquid 1 having a high electric resistivity can be generated.

Note that, a characteristic of device for producing a fine-bubble-containing liquid 10 according to the present embodiment is that device for producing a fine-bubble-containing liquid 10 does not include an introduction valve for supplying a gaseous matter (for example, air) from outside into liquid 2, a pressurized-dissolution chamber for dissolving the supplied gaseous matter in liquid 2, and a water tank for storing fine-bubble-containing liquid 1 that is generated. Thus, device for producing a fine-bubble-containing liquid 10 can generate fine-bubble-containing liquid 1 with a simple configuration that does not include the introduction valve, the pressurized-dissolution chamber, or the water tank. That is, device for producing a fine-bubble-containing liquid 10 can be reduced in size. In addition, fine-bubble-containing liquid 1 can be generated at low cost. Further, since a gaseous matter is not supplied from outside, incorporation into liquid 2 of impurities contained in air supplied from outside can be inhibited.

Next, a method for producing a fine-bubble-containing liquid using device for producing a fine-bubble-containing liquid 10 will be described. Although the method for producing a fine-bubble-containing liquid that is described here will be described with respect to the method for producing the aforementioned samples D and E, the same production method is used for samples A, B, and C, except for a process relating to adjustment of the concentration of the liquid.

First, discharge valve 15 is opened, and then water supply valve 14 is opened to supply ultrapure water with an electric resistivity of 18 MΩ·cm or more into the circulation piping. Next, pump 11 is driven to fill ultrapure water inside pump 11. Thereafter, pump 11 is stopped, water supply valve 14 and discharge valve 15 are closed, and the inside of hermetically sealed flow channel 18 is filled with ultrapure water. Next, injection valve 17 is opened and a fine bubble generation promoter is injected so that a concentration (content) of a fatty acid or a fat-soluble vitamin becomes 10 ppm to 50 ppm, and a concentration (content) of a hydrocarbon becomes 100 ppm to 400 ppm, and injection valve 17 is then closed and pump 11 is driven once more. By this means, liquid 2 is adjusted (first process), and fine bubbles having a size within a range of 1 nm to 0.8 μm are generated in liquid 2 (second process). Pump 11 is stopped after a fixed time period elapses, and sampling valve 16 is opened to extract the generated fine-bubble-containing liquid 1.

A mechanism by which fine bubbles 4 are generated by nozzle 13 will now be described. Device for producing a fine-bubble-containing liquid 10 according to the present embodiment can generate fine bubbles 4 without introducing a gaseous matter into liquid 2 from outside. Specifically, fine bubbles 4 are generated using a gaseous matter (in the present embodiment, air) that is dissolved in liquid 2. That is, a gaseous matter that dissolves in liquid 2 is used. Therefore, pressurization for raising the solubility of a gaseous matter introduced from outside into liquid 2 or the like is not required.

First, a configuration of nozzle 13 will be described. As illustrated in (a) in FIG. 10, nozzle 13 has inflow portion 13a and a plurality of nozzle pipes 13b. Further, as illustrated in (b) in FIG. 10, the plan-view shape of inflow portion 13a and nozzle pipe 13b is approximately circular. The diameter of nozzle pipe 13b is smaller than the diameter of inflow portion 13a. For example, the diameter of nozzle pipe 13b is within a range of 0.5 mm to 2.0 mm, and nozzle pipe 13b extends along a flow direction of liquid 2. In the present embodiment, five nozzle pipes 13b are provided in nozzle 13.

Inflow portion 13a is connected to first piping 12a. The fine bubble generation promoting liquid flows into nozzle pipes 13b from first piping 12a through inflow portion 13a. Because the fine bubble generation promoting liquid that flowed into nozzle pipes 13b is accelerated, the static pressure of the fine bubble generation promoting liquid that flows through nozzle pipes 13b decreases. As a result, a gaseous matter that had been dissolved in the fine bubble generation promoting liquid (specifically, liquid 2 in the fine bubble generation promoting liquid) becomes supersaturated and separates in the fine bubble generation promoting liquid as bubbles. Fine bubbles 4 are generated by the aforementioned bubbles being jetted out from nozzle pipes 13b. By this means, fine-bubble-containing liquid 1 containing fine bubbles 4 is generated. The concentration of fine bubbles 4 generated at nozzle 13 is adjusted, for example, by adjusting a pressure with which pump 11 pumps out liquid 2, and a diameter of nozzle pipe 13b.

Note that, the concentration of fine bubbles 4 can be increased by causing the generated fine-bubble-containing liquid 1 to flow into nozzle 13 once more via pump 11. By causing the generated fine-bubble-containing liquid 1 to circulate until a prescribed concentration of fine bubbles 4 is reached, fine-bubble-containing liquid 1 having a prescribed concentration of fine bubbles 4 can be generated. Further, device for producing a fine-bubble-containing liquid 10 according to the present embodiment does not include a water tank for storing generated fine-bubble-containing liquid 1. Therefore, fine-bubble-containing liquid 1 that is generated by being caused to jet out from nozzle 13 is circulated by flowing into pump 11 through second piping 12b.

Subsequently, pump 11 is stopped after a fixed time period passes, and sampling valve 16 is opened to extract generated fine-bubble-containing liquid 1. For example, by causing fine-bubble-containing liquid 1 to circulate for approximately 15 minutes, fine-bubble-containing liquid 1 containing fine bubbles 4 of a concentration of the order of 10⁹/ml can be generated.

As described above, in accordance with the present embodiment, the method for producing a fine-bubble-containing liquid includes: a first process of generating a liquid (for example, fine bubble generation promoting liquid) by adding one of a fatty acid and a fat-soluble vitamin to water, the fatty acid comprising only carbon, oxygen, and hydrogen; and a second process of generating fine bubbles each having a size within a range of 1 nm to 800 nm, inclusive, in the liquid, without introducing a gaseous matter from outside.

According to this production method fine-bubble-containing liquid 1 that can contain a sufficient concentration of fine bubbles 4 over an extended period of time can be easily produced.

In addition, in the method for producing a fine-bubble-containing liquid, a second process is a process that is conducted by jetting out liquid within hermetically sealed flow channel 18 from nozzle 13.

By this means, fine bubbles 4 can be generated using a gaseous matter which is dissolved in water, and without introducing a gaseous matter from outside. Therefore, fine-bubble-containing liquid 1 can be produced easily and economically.

Further, in the method for producing a fine-bubble-containing liquid, the first process includes a process in which water is supplied to hermetically sealed flow channel 18, the water is caused to circulate and, together therewith, a part of the water is discharged, and a process in which a fatty acid or a fat-soluble vitamin and a hydrocarbon are added into hermetically sealed flow channel 18 until reaching a predetermined amount.

By this means, fine-bubble-containing liquid 1 of stable quality can be produced.

Furthermore, in the adding into the hermetically sealed flow channel, (i) the fatty acid or fat-soluble vitamin, and (ii) the hydrocarbon are added into the hermetically sealed flow channel until a concentration (percentage content) of the fatty acid or the fat-soluble vitamin reaches a concentration within a range of 10 ppm to 50 ppm, inclusive, and a concentration (percentage content) of the hydrocarbon reaches a concentration within a range of 100 ppm to 400 ppm, inclusive.

By this means, production of fine-bubble-containing liquid 1 containing fine bubbles 4 having a size within a range of 1 nm to 0.8 μm at a concentration of 1×10⁹ per ml or more, and which can maintain that concentration of fine bubbles 4 for 30 days or more is facilitated.

Furthermore, in the method for producing a fine-bubble-containing liquid, the water is ultrapure water with an electric resistivity of 18 MΩ·cm or more.

By this means, it is possible to produce fine-bubble-containing liquid 1 in which a content of ions such as metal ions and halogen ions is small, and it is possible to produce highly reliable semiconductor devices by using that fine-bubble-containing liquid 1 as a cleaning fluid.

Further, a configuration of device for producing a fine-bubble-containing liquid 10 is an extremely simple configuration in which the principal parts are only pump 11 for circulating liquid, the circulation piping, and nozzle 13 that is arranged downstream of pump 11. For example, device for producing a fine-bubble-containing liquid 10 includes water supply valve 14 that supplies water, circulation piping (for example, first piping 12a and second piping 12b) that is a flow channel for water, pump 11 that pumps the water, injection valve 17 that injects a fine bubble generation promoter (for example, a fatty acid composed of only carbon, oxygen, and hydrogen, or a fat-soluble vitamin, and a hydrocarbon) into the water, and nozzle 13 that generates fine-bubble-containing liquid 1 having fine bubbles 4 from a fine bubble generation promoting liquid composed of water and the fine bubble generation promoter. Further, device for producing a fine-bubble-containing liquid 10 does not include an introduction valve for introducing a gaseous matter from outside into the water.

Thus, because a complicated equipment configuration including equipment such as a high-pressure pump or a gaseous matter supplying device is not necessary, the equipment for producing a fine-bubble-containing liquid can be constituted in an economical manner. Further, device for producing a fine-bubble-containing liquid 10 can be reduced in size.

Note that, the method for producing fine-bubble-containing liquid 1 is not limited to the above described method. For example, a production method that adopts a pressurized dissolution method, a production method that includes a circulation tank for example, or a production method that uses a swirl flow nozzle may also be adopted. For example, in the case of using the swirl flow nozzle, the swirl flow nozzle serves as a fine bubble generator. A different method may also be adopted. It suffices that a method adopted can produce a prescribed concentration of fine bubbles 4 each having a size within a range of 1 nm to 800 nm from a fine bubble generation promoting liquid. Note that, other methods include a method in which fine bubble generation promoting liquid is caused to pass through a porous body having a pore size of μm order at high pressure, and a method in which a gaseous matter is supplied and passed through a porous body. Although it is also possible to use these other methods, these methods require complicated production equipment, and hence the cost associated with the production equipment increases and a production process also becomes complicated.

[1-4. Cleaning of Semiconductor Devices Using Fine-Bubble-Containing Liquid]

Fine-bubble-containing liquid 1 of the present embodiment is particularly useful in a cleaning process during production of semiconductor devices.

In recent years, wiring densities of semiconductor substrates are being made increasingly finer, and there is a need for a cleaning method that can remove a fine foreign matter that adheres between the wiring. In the case of cleaning that is conducted using microbubbles (refers to bubbles having a size within a range of 1 μm to 1000 μm), because a bubble diameter is large, it is difficult to adequately remove the foreign matter that adheres among fine wiring patterns formed on a semiconductor substrate. Further, because dimensions of patterns formed on semiconductor substrates are becoming narrower and aspect ratios are becoming larger, use of high-frequency ultrasonic cleaning or functional water cleaning that is based on two fluids which are existing cleaning techniques will lead to pattern damage, and hence there are expectations that use of fine bubbles will enable semiconductor substrate cleaning that realizes low damage and a high rate of removal. Fine-bubble-containing liquid 1 containing bubbles each having a diameter ϕ of 0.8 μm or less is effective for removing the fine foreign matter from a semiconductor substrate having such fine wiring.

Furthermore, a high degree of cleanness is required for the cleaning water that is used for cleaning semiconductor substrates. In particular, because contamination with ions such as metal ions or halogen ions significantly affects the reliability of semiconductor products, control at the ppt level is required, and thus ultrapure water in which an amount of ion contamination is low, that is, the electric resistivity is high (electric resistivity is 18 MΩ·cm or more), is used. Further, to ensure that the ultrapure water is not contaminated, the liquid-contacting parts such as a pump and pipes are composed of a Teflon (registered trademark) material such as PFA or PTFE. Note that, although the same level of metallic contamination control is also required for cleaning water that contains fine bubbles, even if fine bubbles are generated with an apparatus composed of Teflon (registered trademark) material using ultrapure water, the concentration will be no more than around 1 to 2×10⁸/ml, and thus fine bubbles of a sufficient concentration for cleaning semiconductor substrates cannot be generated. For example, fine-bubble-containing liquid 1 can be generated by an apparatus composed of Teflon (registered trademark) material or the like by using ultrapure water to which a fine bubble generation promoter as described above is added (one example of a fine bubble generation promoting liquid).

Note that, although a method is available which generates fine bubbles by adding a surface-active agent, because the bubble surface potential in the case of a nonionic surface-active agent that does not contain sodium or potassium that influence the characteristics of a semiconductor device is only in the range of −10 mV to −20 mV and is thus a low value compared with an ionic surface-active agent (bubble surface potential in the range of −20 mV to −50 mV), the contaminant adsorption capacity thereof is low and a sufficient cleaning capacity cannot be obtained.

Thus, conventionally it has been difficult to generate a high concentration of fine bubbles which also have a long lifetime, while maintaining the cleanness of ultrapure water.

By utilizing the fine-bubble-containing liquid (in particular, sample E) of the present embodiment, a method can be provided that, with respect to cleaning designed to remove the fine foreign matter from semiconductor substrates, easily generates fine bubbles at a high concentration and which have a long lifetime while maintaining the cleanness of ultrapure water. Further, the bubble surface potential of fine-bubble-containing liquid 1 of the present embodiment is approximately −35 mV, and thus fine-bubble-containing liquid 1 is excellent in contaminant adsorption capacity and has excellent cleaning properties for cleaning semiconductor substrates and the like.

### Embodiment 2

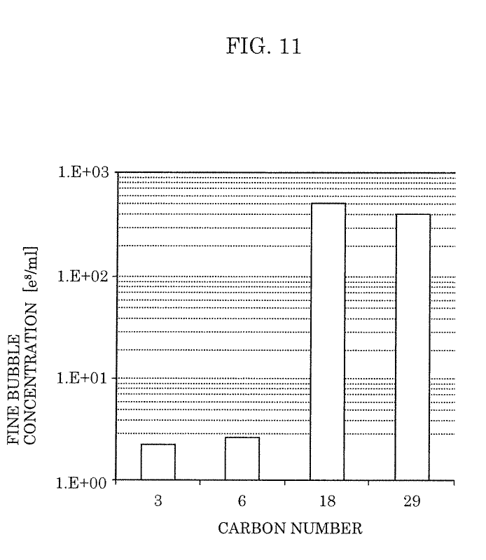

Hereunder, the present embodiment will be described with reference to FIG. 11 to FIG. 19. Note that, the following description centers on points that differ from Embodiment 1, and a description regarding substantially the same configurations or compositions as in Embodiment 1 may be omitted or simplified.

[2-1. Composition of Fine-Bubble-Containing Liquid]

First a composition of a fine-bubble-containing liquid will be described.

The fine-bubble-containing liquid according to the present embodiment contains water, a fat-soluble vitamin or a fatty acid composed only of carbon, oxygen, and hydrogen, and fine bubbles. That is, in the present embodiment the fine-bubble-containing liquid does not contain hydrocarbon.

The water contains a fatty acid or a fat-soluble vitamin (hereunder, also referred to as an “additive agent”) and fine bubbles. The water is, for example, distilled water or ion-exchanged water. In the present embodiment, similarly to Embodiment 1, ultrapure water with an electric resistivity of 18 MΩ·cm or more is used as the water. Note that, hereunder ultrapure water is described as an example of the water. Further, the water may also be referred to as “ultrapure water”.

Fine-bubble-containing liquid is generated by using a predetermined method to generate fine bubbles in water into which an additive agent (a fatty acid or a fat-soluble vitamin) is added (dispersed) (hereunder, such water is also referred to as a “fine bubble generation promoting liquid”). For example, the fine-bubble-containing liquid is water that includes an additive agent and fine bubbles.

The fatty acid composed of only carbon, oxygen, and hydrogen or the fat-soluble vitamin is an additive agent that is added to the water (which is a solvent; ultrapure water in the present embodiment). A characteristic of the present embodiment is that substantially only the fatty acid composed of carbon (C), oxygen (O), and hydrogen (H), or the fat-soluble vitamin is added to the water. That is, the only substance that is intentionally added to the water is the fatty acid or the fat-soluble vitamin. For example, a hydrocarbon is not added. Note that, a substance that is added unintentionally is, for example, an eluted substance (for example, an organic substance) that is eluted into the water from device for producing a fine-bubble-containing liquid 10 (see FIG. 10) during production of the fine-bubble-containing liquid.

Further, a fatty acid compound containing other elements in addition to carbon, oxygen, and hydrogen (for example, sodium deoxycholate that contains sodium: C₂₄H₃₉NaO₄) is not used as the additive agent. In other words, a fatty acid simple substance or a fat-soluble vitamin simple substance is used as the additive agent according to the present embodiment, and a compound such as a fatty acid compound is not used. For example, a surface-active agent is not used as the additive agent. Note that, the details of the fatty acid and the fat-soluble vitamin are described later.

The fine bubbles are bubbles that are present in the fine-bubble-containing liquid, and for example, are air. Although described in detail later, because the fine-bubble-containing liquid contains fine bubbles, the fine-bubble-containing liquid can obtain a cleaning capacity and the like.

As described above, the term “fine bubbles” refers to ultrafine bubbles (nanobubbles) that are bubbles having a nano-order size. Further, in general, when the size of bubbles is small, the lifetime of the bubbles in the fine-bubble-containing liquid is lengthens. Therefore, from a viewpoint of prolonging the lifetime of the fine bubbles also, it is preferable that the size of the fine bubbles is within the range of 1 nm to 200 nm. Note that, this similarly applies with respect to the case where the fine-bubble-containing liquid contains a hydrocarbon that is described in Embodiment 1.

[2-1-1. Types of Additive Agent] Next, the additive agent that is added to water will be described with reference to FIG. 11 and FIG. 12.

FIG. 11 is a view illustrating a relation between a carbon number of an additive agent and a concentration of fine bubbles according to the present embodiment. FIG. 12 is a view illustrating an example of fatty acids and a fat-soluble vitamin according to the present embodiment.

The higher the concentration of fine bubbles contained in the ultrapure water is, the higher a performance as a fine-bubble-containing liquid tends to be. For example, with respect to industrial products, the capacity to remove oxides, an oil content, or the like increases. That is, the cleaning capacity increases. Therefore, it is favorable if the concentration of fine bubbles contained in the ultrapure water is high. Note that, in the conventional method (for example, PTL 1), the concentration of bubbles is 1×10⁷ per ml or higher.

In the present embodiment, as illustrated in FIG. 11, there is a tendency for the concentration of fine bubbles to increase as the carbon number of the additive agent (fatty acid or fat-soluble vitamin) increases. In a case where the carbon number is 18 or 29, the concentration of fine bubbles increases by a large margin (100-fold or more) compared with a case where the carbon number is 3 or 6. The concentration of fine bubbles in a case where the carbon number is 18 is approximately 5×10¹⁰ per ml, while the concentration of fine bubbles in a case where the carbon number is 29 is approximately 4×10¹⁰ per ml. The is because a fatty acid having the carbon number of 18 or 29 is a long-chain fatty acid (fatty acid having a carbon number of 12 or higher) and it is easy for the long chain thereof to become entangled with fine bubbles. Therefore, from a viewpoint of increasing the concentration of fine bubbles, it is favorable for the fatty acid that is used as the additive agent to be the long-chain fatty acid.

Further, the fine bubble generation promoting liquid is generated by adding the fatty acid or the fat-soluble vitamin into ultrapure water. Therefore, from a viewpoint of generating the fine bubble generation promoting liquid, it is favorable if the fatty acid or fat-soluble vitamin is easily added to water (in the present embodiment, ultrapure water). That is, it is favorable if the fatty acid or fat-soluble vitamin is a liquid at a room temperature (for example, a temperature within a range of 18° C. to 25° C.).

Fatty acids include saturated fatty acids and unsaturated fatty acids. For example, in a case where the fatty acid is the saturated fatty acid, at a room temperature the fatty acid is generally a gas if the carbon number thereof is 4 or less, and is generally a solid if the carbon number thereof is 13 or more. Therefore, in a case where the fatty acid is the saturated fatty acid, it is preferable that the carbon number thereof is within a range of 5 to 12. For example, saturated fatty acids that have the carbon number within the range of 5 to 12 include caprylic acid and nonanoic acid.

Further, for example, in a case where the fatty acid is the unsaturated fatty acid, in general if the carbon number thereof is 11 or less the fatty acid will not be a liquid at a room temperature, or may be a liquid at a room temperature but will have a low boiling point and will vaporize during generation of the fine-bubble-containing liquid. Therefore, in a case where the fatty acid is the unsaturated fatty acid it is preferable that the carbon number thereof is 12 or more. For example, unsaturated fatty acids that have the carbon number of 12 or more include palmitoleic acid, oleic acid, linoleic acid, α-linolenic acid, and arachidonic acid.

Note that, as described above, in a case where the additive agent is a liquid at a room temperature but has a low boiling point, in some cases the additive agent vaporizes during production of the fine-bubble-containing liquid. Because the additive agent cannot become involved with (adsorb) fine bubbles, the generated fine bubbles immediately disappear (dissolve in the ultrapure water). Therefore, for example, the boiling point of the additive agent is preferably 100° C. or higher.

The fat-soluble vitamin may also be used as the additive agent. For example, among vitamins A, D, E, and K which are fat-soluble vitamins, a vitamin that is a liquid at a room temperature may be used. Examples of a fat-soluble vitamin that is a liquid at a room temperature include α-tocopherol.

Hereinafter, in a case where the additive agent is not specified, results for a case where oleic acid is used as the additive agent are described.

Note that, a fatty acid that has a carbon number of 3 is generally a gas. Because it is difficult to add a gas to water, the data for the fatty acid having the carbon number of 3 that is shown in FIG. 11 is not data that was obtained using a fatty acid, but is data for a case where, for comparison, a different substance (having a carbon number of 3) composed of carbon, hydrogen, and oxygen was used.

[2-1-2. Concentration of Additive Agent]

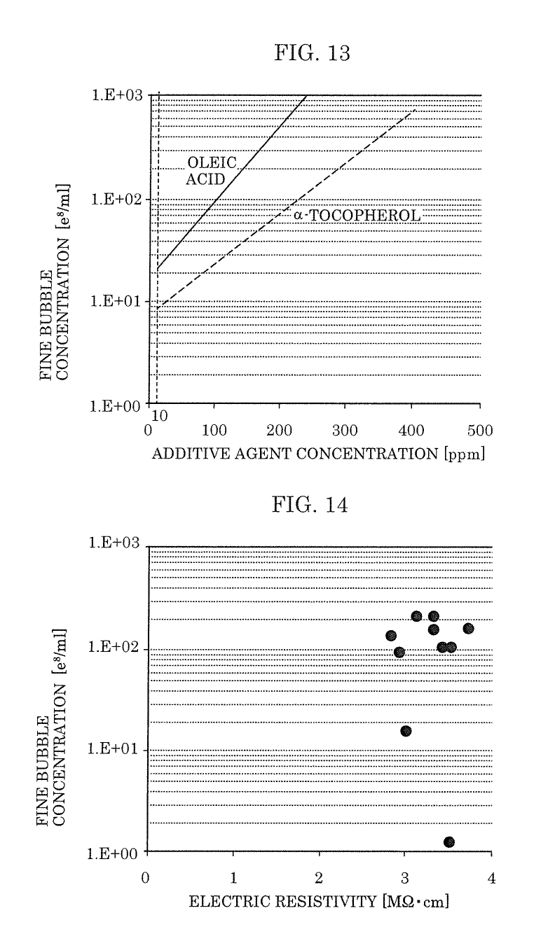

Next, the concentration of the additive agent that is added to the ultrapure water will be described with reference to FIG. 13. Note that the term “concentration of fine bubbles” in FIG. 13 refers to the concentration of bubbles having a size within a range of 1 nm to 200 nm among the bubbles contained in the fine-bubble-containing liquid. The fine-bubble-containing liquid may also contain bubbles having a size that is less than 1 nm or greater than 200 nm.

FIG. 13 is a view illustrating a relation between a concentration of the additive agent and a concentration of fine bubbles according to the present embodiment. Results for cases where oleic acid and α-tocopherol are used as examples of the additive agent are illustrated in FIG. 13. The solid line in FIG. 13 indicates the result for a case where oleic acid was added, and the dashed line in FIG. 13 indicates the result for a case where α-tocopherol was added. Note that, in FIG. 13 the concentration of fine bubbles is shown for a range in which the concentration of the additive agent is from 10 ppm to 400 ppm.

As illustrated in FIG. 13, it is found that as the additive agent concentration is increased, the concentration of fine bubbles also increases. This tendency is the same for both oleic acid that is the fatty acid and α-tocopherol that is the fat-soluble vitamin. In the present embodiment, when the concentration of the additive agent is within the range from 10 ppm to 400 ppm, the concentration of the additive agent and the concentration of fine bubbles are in a proportional relation. For example, in a case where the additive agent is oleic acid, when the concentration of oleic acid is 10 ppm, the concentration of fine bubbles is approximately 2.3×10⁹ per ml. For example, in a case where the additive agent is α-tocopherol, when the concentration of α-tocopherol is 10 ppm, the concentration of fine bubbles is approximately 8×10⁸ per ml. That is, if the concentration of the additive agent is 10 ppm or more, the fine-bubble-containing liquid can be generated that contains a higher concentration of fine bubbles than a fine-bubble-containing liquid according to the conventional methods.

Further, the higher the concentration of fine bubbles is, the higher the performance (for example, cleaning performance) of the fine-bubble-containing liquid becomes. Therefore, the higher concentration of fine bubbles is better. For example, the concentration of fine bubbles may be 1×10⁹ per ml or more. For example, in a case where the additive agent is α-tocopherol, the concentration of α-tocopherol is approximately 20 ppm or more. In addition, for example, the concentration of fine bubbles is more preferably 3×10⁹ per ml or more. Thus, the fine-bubble-containing liquid has a higher cleaning capacity. In this case, for example, when the additive agent is α-tocopherol, the concentration of α-tocopherol is approximately 120 ppm or more.

It is favorable to set the upper limit of the additive agent concentration, for example, to a value that does not exceed a critical micelle concentration of the additive agent. If an additive agent is added in an amount exceeding the critical micelle concentration, the additive agent will form micelles. If micelles are formed, the additive agent will not adsorb fine bubbles. As a result it will be difficult to raise the concentration of fine bubbles. Although not illustrated in the drawings, it was confirmed by an experiment that even if the additive agent concentration is made 600 ppm, micelles do not form. Therefore, taking into consideration the critical micelle concentration, the additive agent concentration is preferably not more than 600 ppm.

On the other hand, when performing precision cleaning such as cleaning of semiconductors using the fine-bubble-containing liquid, if the concentration of the additive agent is high it is possible that the additive agent itself will become a contamination source. Therefore, it is favorable for the concentration of the additive agent to be a concentration of a level such that the additive agent does not become the contamination source. The concentration of the additive agent at which the additive agent does not become the contamination source is, for example, 400 ppm or less.

Further, an upper limit of the concentration of the additive agent may be set from a viewpoint of efficiently generating fine bubbles. In FIG. 13, the concentration of the additive agent and the concentration of fine bubbles are in a proportional relation. That is, as the concentration of the additive agent is increased, the concentration of fine bubbles also increases in proportion thereto. However, from around the vicinity where the additive agent concentration exceeds 400 ppm, the amount of increase in the concentration of fine bubbles relative to the amount of increase in the additive agent concentration becomes less in comparison to a case where the additive agent concentration is not more than 400 ppm. In other words, when the concentration of the additive agent exceeds 400 ppm, the slopes of the straight lines in FIG. 13 become gentle. That is, even if the additive agent is increased, it is difficult to efficiently increase the concentration of fine bubbles. Therefore, if the concentration of the additive agent is 400 ppm or less, a high concentration of fine bubbles can be generated efficiently.

As illustrated in FIG. 13, with respect to the difference between oleic acid and α-tocopherol, when oleic acid is added, an added amount of the additive agent with respect to the concentration of fine bubbles that are generated can be reduced relative to when α-tocopherol is added. Further, as illustrated in FIG. 11, by adopting the fatty acids having the carbon number of 18 which include oleic acid as the additive agent, the added amount of the additive agent can be reduced.

Furthermore, with respect to the difference between oleic acid and α-tocopherol, although the tendencies are the same, the slopes of the straight lines are different. That is, the relation between the concentration of the additive agent and the concentration of fine bubbles differs depending on the kind of the fatty acid or the fat-soluble vitamin. Therefore, the additive agent may be appropriately selected so that the concentration of the additive agent is, for example, within the range of 10 ppm to 400 ppm based on the concentration of the fine bubbles that are generated.

[2-2. Detailed Description of Fine-Bubble-Containing Liquid]

First, a description regarding the electric resistivity of the fine-bubble-containing liquid and the concentration of fine bubbles will be given with reference to FIG. 14. Note that, the term “concentration of fine bubbles” in FIG. 14 refers to the concentration of bubbles having a size within a range of 1 nm to 200 nm among the bubbles contained in the fine-bubble-containing liquid. The fine-bubble-containing liquid may also contain bubbles having a size that is less than 1 nm or greater than 200 nm.

FIG. 14 is a view that illustrates a relation between an electric resistivity and a concentration of fine bubbles of the fine-bubble-containing liquid according to the present embodiment. As described above, in the present embodiment, ultrapure water with an electric resistivity of 18 MΩ·cm or more and which substantially does not contain impurities is used as the water. FIG. 14 illustrates the relation between the electric resistivity and the concentration of fine bubbles of the fine-bubble-containing liquid in which oleic acid is added to the ultrapure water.

As illustrated in FIG. 14, even when the concentration of fine bubbles changes, the electric resistivity of the fine-bubble-containing liquid is approximately constant. Specifically, when the concentration of fine bubbles is within a range of 1.2×10⁸ per ml to 2.2×10¹⁰ per ml, the electric resistivity of the fine-bubble-containing liquid is approximately 3 to 4 MΩ·cm. This means that even if the concentration of fine bubbles (that is, the concentration of the additive agent) increases, an amount of impurities contained in the fine-bubble-containing liquid is small. Based on FIG. 13 it is found that, when the additive agent is oleic acid, the time that the concentration of fine bubbles is 2.2×10¹⁰ per ml is when the concentration of oleic acid is approximately 150 ppm. For example, when the concentration of oleic acid that is added is not more than 150 ppm, the electric resistivity of the fine-bubble-containing liquid containing oleic acid is approximately constant irrespective of the concentration thereof.

The electric resistivity of the fine-bubble-containing liquid is preferably 1 MΩ·cm or more that is the electric resistivity of what is commonly called “pure water”. Thus, the amount of impurities contained in the fine-bubble-containing liquid is small, and it is difficult for the additive agent to become a contamination source. Hence, the fine-bubble-containing liquid according to the present embodiment can be used for uses in which a cleaning fluid of high purity is required, such as cleaning (precision cleaning) of semiconductors. Further, since the fine-bubble-containing liquid according to the present embodiment contains fine bubbles, it can be said that the cleaning capacity thereof is higher than in a case of performing cleaning using only ultrapure water.

Note that, as described above, FIG. 14 illustrates results for a case in which the ultrapure water is used as the water. In a case where impurities are contained in the water (impurities are substantially contained), the results will be different from FIG. 14.

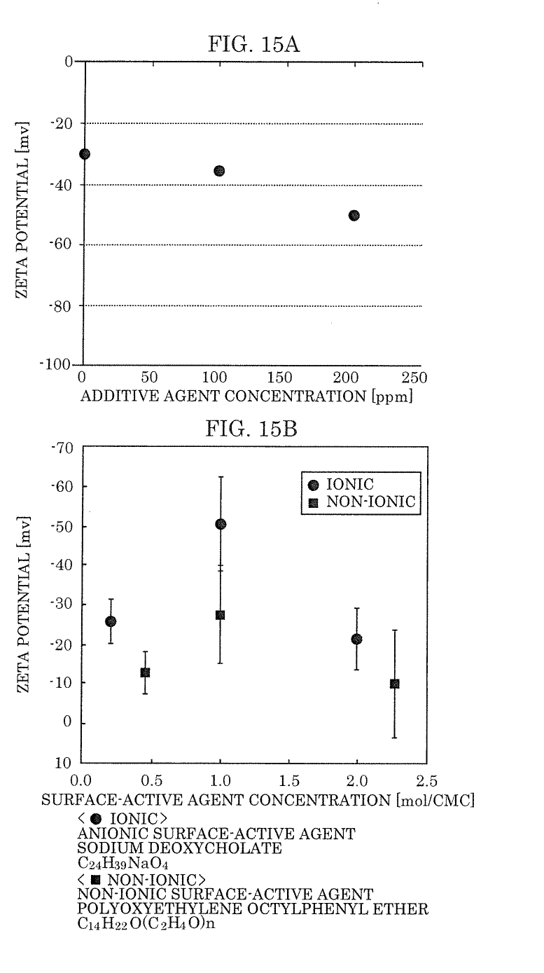

Next, a relation between a concentration of the fatty acid and a zeta potential of fine bubbles will be described with reference to FIG. 15A and FIG. 15B.

FIG. 15A is a view illustrating a relation between a concentration of the fatty acid and the zeta potential according to the present embodiment. FIG. 15B is a view illustrating a relation between a concentration of a surface-active agent and a zeta potential of bubbles according to Conventional Examples. Note that, as the Conventional Examples, sodium deoxycholate (C₂₄H₃₉NaO₄) that is an ionic surface-active agent (anionic) and polyoxyethylene octylphenyl ether (C₁₄H₂₂O(C₂H₄O)n) that is a non-ionic surface-active agent are used. Further, although the ordinate axis in FIG. 15A and FIG. 15B represents the zeta potential, in FIG. 15A the zeta potential is represented so as to increase toward the lower side (side on which the ordinate axis intersects with the abscissa axis) of the ordinate axis, while in FIG. 15B the zeta potential is represented so as to increase toward the upper side (opposite side to the side on which the ordinate axis intersects with the abscissa axis) of the ordinate axis.

The surface of the fine bubbles is charged to a negative potential in the water (in the present embodiment, ultrapure water). The term “zeta potential” refers to a potential at a sliding surface of the fine bubbles. If the zeta potential is high (the negative potential at the sliding surface of the fine bubbles is high), it is easy for the fine bubbles to adsorb substances that are charged to a positive potential. For example, since oxides, an oil content, and the like are positively charged, if the zeta potential of the fine bubbles is high, it is easy for the fine bubbles to adsorb impurities such as the oxides and the oil content as well as dirt. That is, when the zeta potential is high, the cleaning capacity of the fine-bubble-containing liquid increases. Therefore, from a viewpoint of the cleaning capacity, it is favorable for the zeta potential of the fine bubbles to be high. Further, when the zeta potential is high, a repulsive force between fine bubbles is strong and the dispersion properties of the fine bubbles increase. Conversely, if the absolute value of the zeta potential is low, the fine bubbles are liable to aggregate. Therefore, from a viewpoint of the dispersion properties of the fine bubbles also, it is favorable for the zeta potential to be high.

As illustrated in FIG. 15A, when the concentration of the fatty acid (in the present embodiment, oleic acid) is increased, the zeta potential becomes higher. Specifically, the zeta potential is approximately −30 mV in a case where the fatty acid concentration is 0 ppm, and the zeta potential is approximately −35 mV in a case where the fatty acid concentration is 100 ppm. When the fatty acid concentration is 100 ppm, the zeta potential is higher in comparison with when the fatty acid concentration is 0 ppm, although only by a small amount. Further, when the fatty acid concentration is 200 ppm, the zeta potential is approximately −50 mV. The zeta potential increases between the fatty acid concentrations of 100 ppm and 200 ppm. That is, the zeta potential can be increased by making the concentration of the fatty acid 100 ppm or more.

Although the results for the fatty acid concentrations up to 200 ppm are shown in FIG. 15A, it was found that, up to the fatty acid concentration of around 600 ppm, the zeta potential increases when the concentration of the additive agent is increased. Note that it is considered that the reason the zeta potential is approximately −30 mV when the fatty acid concentration is 0 ppm is that organic substances from the production equipment (for example, device for producing a fine-bubble-containing liquid 10) made of the Teflon (registered trademark) material that is used for generating the fine-bubble-containing liquid dissolved in the fine-bubble-containing liquid.

As illustrated in FIG. 15B, when the concentration of ionic and non-ionic surface-active agents is 1.0 mol/CMC (critical micelle concentration), the zeta potential is high. Specifically, the zeta potential of the ionic surface-active agent is approximately −50 mV, and the zeta potential of the non-ionic surface-active agent is approximately −27 mV. However, the surface-active agent forms micelles if the concentration thereof is 1.0 mol/CMC or higher. That is, 1.0 mol/CMC is the critical micelle concentration of the surface-active agent. If the surface-active agent forms micelles, the surface-active agent no longer contributes to an increase in the zeta potential and the generation of bubbles. Therefore, it becomes difficult to increase the zeta potential of fine bubbles and also to generate a high concentration of bubbles in the water. That is, in the case of the surface-active agent, an amount of surface-active agent that can be added to the water is limited, and it is difficult to generate fine-bubble-containing liquid that has a high zeta potential and which contains a high concentration of fine bubbles. This applies similarly to the ionic and non-ionic surface-active agents.

Further, among the ionic and non-ionic surface-active agents, while it is better to use the ionic surface-active agent in order to increase the zeta potential more, the ionic surface-active agent can become the cause of contamination when performing precision cleaning such as semiconductor cleaning. Therefore, when performing precision cleaning, it is not preferable to use the ionic surface-active agent. Further, when performing precision cleaning, although the non-ionic surface-active agent can be used, the zeta potential is low, as illustrated in FIG. 15B. Specifically, at the concentration of 0.5 mol/CMC, the zeta potential is approximately −13 mV, which is low compared with the fine bubbles according to the present embodiment. In other words, a cleaning effect of the bubble-containing liquid that contains the non-ionic surface-active agent is low compared with the fine-bubble-containing liquid according to the present embodiment.

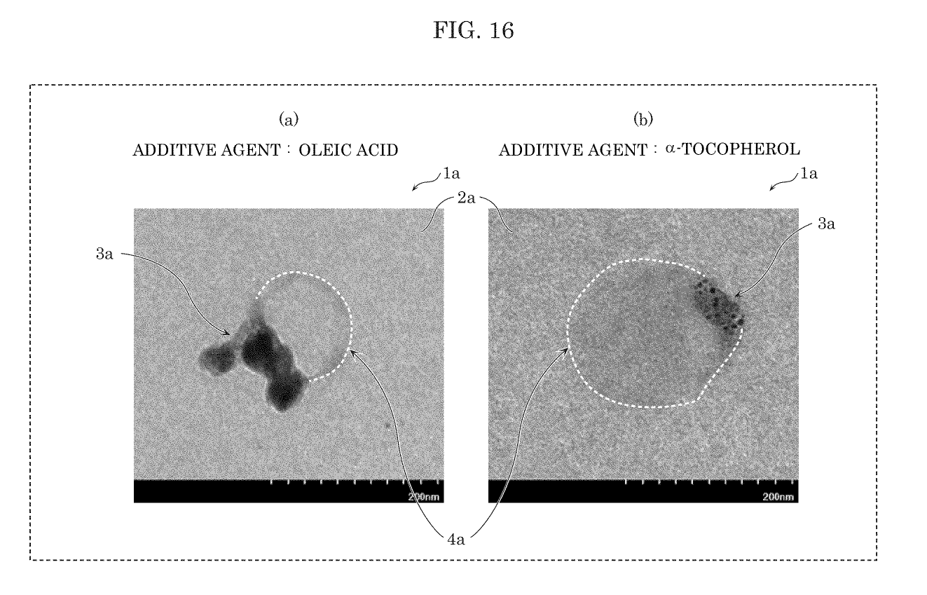

Next, a structure of the fine bubbles will be described with reference to FIG. 16.

FIG. 16 is a view showing a TEM image of fine bubble 4a which is adsorbed on additive agent 3a of fine-bubble-containing liquid 1a according to the present embodiment. In (a) in FIG. 16, the TEM image of fine bubble 4a in a case where the concentration of additive agent 3a (in the present embodiment, oleic acid) is 200 ppm is shown. In (b) of FIG. 16, the TEM image of fine bubble 4a in a case where the concentration of additive agent 3a (in the present embodiment, α-tocopherol) is 400 ppm is shown. Note that the size of fine bubble 4a in each of these images is approximately 100 to 150 nm.

In (a) in FIG. 16, a region that is completely filled with black indicates additive agent 3a. Further, the white dashed line shows a contour of fine bubble 4a. Similarly, in (b) in FIG. 16, a region that is completely filled with black indicates additive agent 3a, and the white dashed line shows the contour of fine bubble 4a. As illustrated in (a) in FIG. 16 and (b) in FIG. 16, it is found that additive agent 3a adsorbs fine bubble 4a. That is, fine-bubble-containing liquid 1a according to the present embodiment has a structure in which additive agent 3a adsorbs fine bubbles 4a (additive agent 3a and fine bubbles 4a contact each other).

When fine bubble 4a is in the state of a simple substance, fine bubble 4a cannot maintain that state and consequently disappears (for example, dissolves in water). For example, even if fine bubbles 4a are generated using ultrapure water 2a that does not contain additive agent 3a, generated fine bubbles 4a will immediately disappear. It is considered that in fine-bubble-containing liquid 1a according to the present embodiment, as illustrated in (a) and (b) of FIG. 16, the disappearance of fine bubbles 4a is inhibited to a greater extent by additive agent 3a adsorbing fine bubbles 4a. In other words, because additive agent 3a adsorbs fine bubbles 4a, it is possible to further prolong the lifetime of fine bubbles 4a.

The number of additive agents 3a (a fatty acid or a fat-soluble vitamin) that partially cover the surface of fine bubble 4a is not particularly limited. It suffices that fine bubble 4a is partially covered with at least one additive agent 3a. By this means, disappearance of fine bubble 4a can be inhibited. Note that, in (a) of FIG. 16 a case is illustrated in which a plurality of oleic acids adsorb fine bubble 4a, and in (b) in FIG. 16 a case is illustrated in which a plurality of α-tocopherols adsorb fine bubble 4a.

Further, for example, additive agent 3a may cover 10% to 70% of a surface area of fine bubble 4a. Note that, it is not necessary for all of fine bubbles 4a contained in fine-bubble-containing liquid 1a to be covered with additive agent 3a.

Additive agent 3a has hydrophobicity. Further, fine bubble 4a is formed, for example, of air, and the principal components of air are nitrogen and oxygen. Nitrogen and oxygen have hydrophobicity. Therefore, additive agent 3a and fine bubbles 4a which are formed of air have compositions which facilitate contact between additive agent 3a and fine bubbles 4a.

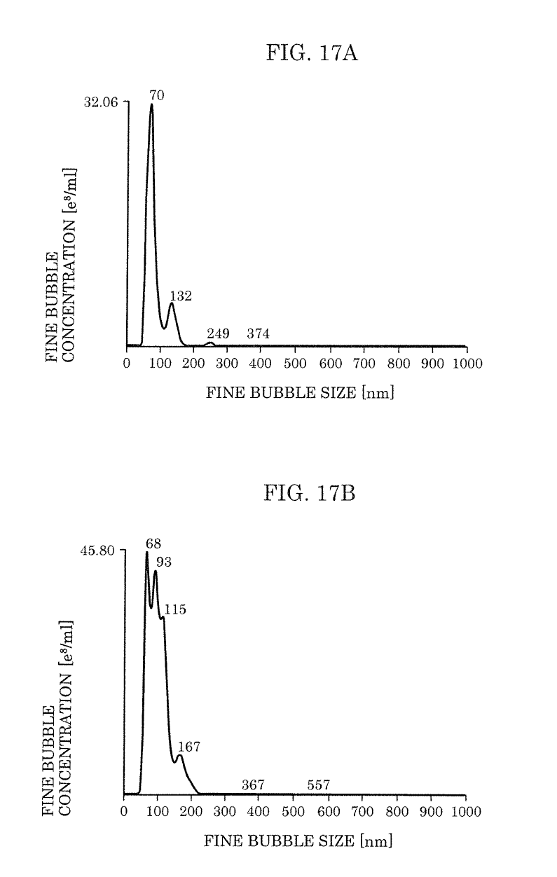

Next, a size distribution of fine bubbles 4a will be described with reference to FIG. 17A and FIG. 17B.

FIG. 17A is a view illustrating the size distribution of fine bubbles 4a in a case where oleic acid was added according to the present embodiment. FIG. 17B is a view illustrating the size distribution of fine bubbles 4a in a case where α-tocopherol was added according to the present embodiment. The concentrations of fine bubbles 4a mentioned above were obtained by measuring the concentration of bubbles having a size within a range of 1 nm to 800 nm in FIG. 17A and FIG. 17B.

As illustrated in FIG. 17A and FIG. 17B, it is found that mainly fine bubbles 4a having a size within the range of 1 nm to 200 nm are contained in fine-bubble-containing liquid 1a. In FIG. 17A, the proportion of bubbles having a size of approximately 70 nm is large. Further, in FIG. 17B the proportion of bubbles having a size within the range of 68 nm to 115 nm is large. As described above, the term “fine bubbles 4a” refers to bubbles having a size within the range of 1 nm to 800 nm. That is, the bubbles contained in fine-bubble-containing liquid 1a are mainly fine bubbles 4a (nanobubbles). For example, the proportion of fine bubbles 4a having a size within a range of 1 nm to 200 nm among bubbles contained in fine-bubble-containing liquid 1a is 90% or more. More preferably, the proportion is 95% or more. By having a high proportion of fine bubbles 4a, fine-bubble-containing liquid 1a can exert a greater effect in precision cleaning.

Further, it is found that bubbles having a size exceeding 200 nm are contained in fine-bubble-containing liquid 1a. Note that, it is considered that by additive agent 3a adsorbing the bubbles having a size exceeding 200 nm, it is possible to prolong the lifetime of those bubbles, similarly to the case of fine bubbles 4a. That is, additive agent 3a according to the present embodiment makes it possible to prolong the lifetime of bubbles, regardless of the size of the bubbles.

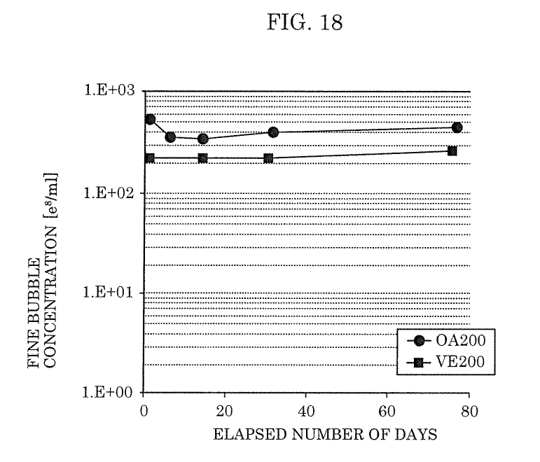

Next, the lifetime of fine bubbles 4a that are described above will be described with reference to FIG. 18.

FIG. 18 is a view illustrating the lifetime of fine bubbles 4a according to the present embodiment. Specifically, the concentration of fine bubbles 4a in generated fine-bubble-containing liquid 1a was measured per elapsed time period. In FIG. 18, reference character OA200 denotes a result for a case where oleic acid was added as additive agent 3a, and reference character VE200 denotes a result for a case where α-tocopherol was added as additive agent 3a. Note that, the concentration of oleic acid and α-tocopherol was 200 ppm, respectively.

As illustrated in FIG. 18, after generating fine-bubble-containing liquid 1a, there was no significant change in the concentration of fine bubbles 4a in fine-bubble-containing liquid 1a even after 70 days or more had passed. This applied similarly for both the case where additive agent 3a was oleic acid and the case where additive agent 3a was α-tocopherol. For example, in the case of OA200 (oleic acid), the concentration of fine bubbles 4a in fine-bubble-containing liquid 1a was approximately 5×10¹⁰ per ml immediately after (0 days) generation of fine-bubble-containing liquid 1a, and was approximately 4.5×10¹⁰ per ml after approximately 75 days had passed. Further, in the case of VE200 (α-tocopherol), the concentration of fine bubbles 4a in fine-bubble-containing liquid 1a was approximately 2.2×10¹⁰ per ml immediately after generation of fine-bubble-containing liquid 1a, and was approximately 2.7×10¹⁰ per ml after 75 days had passed. Thus, the concentration of fine bubbles 4a could be maintained at a high state irrespective of whether additive agent 3a was oleic acid or α-tocopherol.

Further, additional generation of bubbles was not performed during the period from the generation until 75 days passed. That is, it is considered that during the period from immediately after the generation until 75 days passed, the concentration of fine bubbles 4a is stable, and the state of fine bubbles 4a that were contained at the time fine-bubble-containing liquid 1a was generated could be maintained even after 75 days passed. Therefore, the lifetime of fine bubbles 4a is 75 days or more. From a practical viewpoint, preferably the lifetime of fine bubbles 4a is 30 days or more. Note that, the phrase “lifetime of fine bubbles 4a” refers to, for example, when taking the concentration of fine bubbles 4a immediately after generation of fine-bubble-containing liquid 1a as a reference value, the time period until the concentration of fine bubbles 4a become equal to or less than half of the reference value.

[2-3. Method for Producing a Fine-Bubble-Containing Liquid]

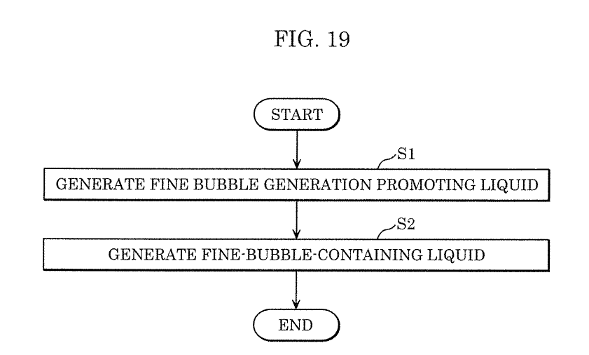

Next, a method for producing the aforementioned fine-bubble-containing liquid 1a will be described with reference to FIG. 19. Note that, the production device has a same configuration as device for producing a fine-bubble-containing liquid 10 that is described in Embodiment 1, and hence a description thereof is omitted hereunder. In the present embodiment, the additive agent that is injected from injection valve 17 is different from Embodiment 1. In the present embodiment, a fatty acid composed of only carbon, oxygen or a fat-soluble vitamin is injected into the water from injection valve 17. That is, hydrocarbon is not injected from injection valve 17. Note that, in this case, the fatty acid or fat-soluble vitamin is an example of a fine bubble generation promoter.

FIG. 19 is a flowchart illustrating the flow of the method for producing fine-bubble-containing liquid 1a according to the present embodiment.

In a state in which the inside of device for producing a fine-bubble-containing liquid 10 is filled with ultrapure water 2a, fine bubble generation promoting liquid is generated by adding additive agent 3a to ultrapure water 2a through injection valve 17 (additive agent 3a is added to ultrapure water 2a) (S1). In the present embodiment, since ultrapure water 2a is supplied directly through water supply valve 14 from an ultrapure water generating device (not illustrated), the electric resistivity of filled ultrapure water 2a is 18 MΩ·cm or more. Further, additive agent 3a is the fatty acid or the fat-soluble vitamin.

Fine bubbles 4a are generated by the generated fine bubble generation promoting liquid that is jetted out from nozzle 13 (specifically, nozzle pipes 13b). It becomes possible to further prolong the lifetime of generated fine bubbles 4a by adsorption of fine bubbles 4a by additive agent 3a. By this means, fine-bubble-containing liquid 1a containing fine bubbles 4a can be generated (S2).

[2-4. Effects and the Like]

As described above, in accordance with the present embodiment, fine-bubble-containing liquid 1a includes: water; a fatty acid or fat-soluble vitamin, the fatty acid comprising only carbon, oxygen, and hydrogen; and fine bubbles 4a, and fine bubbles 4a each have a size within a range of 1 nm to 800 nm, inclusive.

Thus, because the fatty acid or fat-soluble vitamin (additive agent 3a) that is added to the water (in the present embodiment, ultrapure water 2a) is less liable to form micelles in comparison with a surface-active agent that has been used conventionally, the concentration of fine bubbles 4a can be adjusted more easily by adjusting the concentration of additive agent 3a. Specifically, the concentration of fine bubbles 4a can be increased by increasing the concentration of additive agent 3a. Further, in general, when the size of bubbles in a fine-bubble-containing liquid is small, the lifetime of the bubbles lengthens. In the present embodiment, the fine-bubble-containing liquid mainly contains fine bubbles (nanobubbles) that have a size within a range of 1 nm to 800 nm. Therefore, the lifetime of the bubbles (fine bubbles) contained in the fine-bubble-containing liquid lengthens in comparison to a case where a bubble-containing liquid mainly contains microbubbles or the like. Hence, fine-bubble-containing liquid 1a according to the present embodiment can maintain a high concentration of fine bubbles 4a for an extended period of time.

Further, since fine bubbles 4a have a size within a range of 1 nm to 800 nm, for example, fine-bubble-containing liquid 1a according to the present embodiment can be used for cleaning of minute places such as for cleaning to remove a foreign matter from among wiring patterns of semiconductor substrates.

Furthermore, a concentration of the fatty acid or fat-soluble vitamin is 10 ppm or more.

By this means, the concentration of fine bubbles 4a contained in fine-bubble-containing liquid 1a can be made equal to or higher than a predetermined concentration. That is, fine-bubble-containing liquid 1a having a high cleaning capacity can be generated.

Furthermore, a surface of each of fine bubbles 4a is covered with at least a single one of the fatty acid or covered with at least a single one of the fat-soluble vitamin.

Thus, fine bubble 4a has a structure that contacts additive agent 3a (additive agent 3a adsorbs fine bubble 4a). By having this structure, it is possible to further inhibit the disappearance of fine bubbles 4a. That is, fine-bubble-containing liquid 1a that have a high concentration of fine bubbles 4a for a more prolonged period of time can be generated.

Furthermore, it is possible that fine bubbles 4a each have a size of not more than 200 nm.

By this means, in addition, the lifetime of bubbles (fine bubbles) contained in fine-bubble-containing liquid 1a is lengthened.

Furthermore, a lifetime of fine bubbles 4a is 30 days or more.

Conventionally, the time period of the life (lifetime) of a bubble is equal to or longer than one minute. Therefore, storage of liquids containing bubbles has been difficult.

In contrast, because fine-bubble-containing liquid 1a according to the present embodiment has the fatty acid or the fat-soluble vitamin, a lifetime of 30 days or more is realized. As a result, the generated fine-bubble-containing liquid 1a can be stored. That is, it is possible to produce and store fine-bubble-containing liquid 1a. In addition, because there is also little change in the concentration of fine bubbles 4a during storage, even when fine-bubble-containing liquid 1a is used after being stored, a cleaning effect can be obtained that is of the same level as a cleaning effect obtained when fine-bubble-containing liquid 1a is used immediately after being produced.

Further, in the present embodiment, according to the method for producing fine-bubble-containing liquid 1a, the fatty acid composed of only carbon, oxygen, and hydrogen, or the fat-soluble vitamin is added to water to generate a fine bubble generation promoting liquid (S1). Further, fine bubbles 4a having a size within a range of 1 nm to 800 nm are generated in the generated fine bubble generation promoting liquid, without introducing a gaseous matter from outside (S2). Note that, step S1 is an example of a first process, and step S2 is an example of a second process.

By generating fine bubbles 4a from the fine bubble generation promoting liquid that contains the fatty acid or the fat-soluble vitamin, the fatty acid or the fat-soluble vitamin that is added to the water adsorbs fine bubbles 4a, and thus the disappearance of fine bubbles 4a can be inhibited more. That is, the lifetime of fine bubbles 4a is lengthened. Hence, fine-bubble-containing liquid 1a that can maintain a high concentration of fine bubbles 4a for an extended period of time can be generated. Further, since the gaseous matter is not introduced from outside, a step for dissolving the relevant gaseous matter in the water can be omitted. In addition, in a case where the water is ultrapure water 2a, by not introducing the gaseous matter from outside, the occurrence of a situation in which ultrapure water 2a is exposed to the gaseous matter from outside and incorporates contaminants which the gaseous matter from outside has can be suppressed.

### Application Examples

Hereunder, Application Examples of fine-bubble-containing liquids 1 and 1a described in Embodiments 1 and 2 are described. Note that, usages described hereunder are examples, and usages of fine-bubble-containing liquids 1 and 1a are not limited to the following usages. Further, hereunder, fine-bubble-containing liquids 1 and 1a are also described as “ultrafine bubble water”.

In the conventional fine bubble water, the lifetime of the fine bubbles is short, and the usages thereof are limited. The ultrafine bubble water according to the present embodiment can maintain a high concentration of ultrafine bubbles stably over an extended period of time. For example, use of the ultrafine bubble water according to the present embodiment for medical applications, agricultural applications, cosmetics applications, food applications, beverage applications, sterilization applications, cleaning applications, marine product applications, and the like is expected.

With respect to the medical applications, for example, use of the ultrafine bubble water as an ultrasound contrast agent is expected. The bubbles that are currently in widespread use as an ultrasound contrast agent are microbubbles having a diameter within a range of 1.1 μm to 5 μm. However, because the lifetime of such microbubbles is short, modified albumin or the like is used in order to form a shell for maintaining the bubbles. Therefore, in a case of using microbubbles as the ultrasound contrast agent, it is necessary to take into consideration the discharge of the modified albumin or the like to outside of the body.

When ultrafine bubbles that have the long lifetime are used as the ultrasound contrast agent, a shell for maintaining the bubbles is unnecessary, and therefore consideration need not be given to the discharge of the modified albumin to outside of the body or the like. Further, although microbubbles are not capable of passing through capillary blood vessels, ultrafine bubbles can pass through capillary blood vessels because ultrafine bubbles are smaller in size. That is, it is possible to use ultrafine bubbles as an ultrasound contrast agent for capillary blood vessels. Although a substance containing iodine is already known as the ultrasound contrast agent for capillary blood vessels, this substance cannot be used for people with renal disease or the like. On the other hand, because an ultrasound contrast agent that uses ultrafine bubbles does not contain iodine, it is possible to use the ultrasound contrast agent for people with renal disease or the like.

With respect to the agricultural applications, for example, use of the ultrafine bubble water as water to be given to plants is expected. It has been reported that when ultrafine bubble water is given to the roots of plants the water absorption efficiency improves in comparison with when normal water (for example, water that does not contain bubbles) is given. For example, if ultrafine bubble water in which a functional substance is adhered to ultrafine bubbles is absorbed from the roots, the function can be efficiently imparted to the plant. For example, nutrients such as phosphorus, nitrogen, or vitamins or an insect-repellant component such as leaf alcohol can be added. Further, it is possible to prolong a lifetime of a plant by adhering an antioxidant to ultrafine bubbles.

With respect to the cosmetics applications, for example, use as an emulsifier is expected. Currently, a surface-active agent is used for maintaining the dispersion of water and oil (for emulsification). By using ultrafine bubble water, the dispersion of water and oil is maintained by means of the dispersion properties and the long lifetime of the ultrafine bubbles, and hence the surface-active agent need not be added in order to maintain the dispersion of water and oil. Further, as the additive agent that is added when generating the ultrafine bubble water, it is favorable to select, for example, an additive agent with respect to which a problem does not arise even when used as a cosmetic application.

With respect to the food applications, use as water to be used when producing food is expected. For example, by using the ultrafine bubble water as water that is used when producing jelly or bread, it is possible to change mouthfeel. For example, it is possible to change the mouthfeel by the simple adjustment of changing the concentration of bubbles in the ultrafine bubble water. Further, it is possible to generate the ultrafine bubble water in which an aromatic fatty acid or the like is adhered as an additive agent to the ultrafine bubbles. By using the ultrafine bubble water in question in the production of jelly or bread or the like, it is possible to flavor the jelly or bread or the like. In addition, since the lifetime of the ultrafine bubbles is long, the aroma can be maintained for an extended period of time.

With respect to the beverage applications, for example, use as water that is used in the production of alcohol such as Japanese rice wine is expected. For example, if ultrafine bubble water is used in a brewing process, fermentation is promoted by the dispersion effect thereof, and thus the time required to produce Japanese rice wine or the like can be shortened. Further, by using nitrogen bubbles it is possible to inhibit oxidation of Japanese rice wine or the like.

With respect to the sterilization applications, for example, use for long-term storage of sodium hypochlorite that is used for sterilization and the like is expected. If sodium hypochlorite is stored for an extended period of time as an aqueous solution, the sodium hypochlorite decomposes and changes to a saline solution. In other words, the long-term storage of sodium hypochlorite is difficult. Further, because the sodium hypochlorite is decomposed, uneven dispersion of sodium hypochlorite occurs. Therefore, for example, by causing sodium hypochlorite to adhere to ultrafine bubbles by using a fatty acid or the like that causes sodium hypochlorite and ultrafine bubbles to attach to each other, decomposition of sodium hypochlorite that occurs due to aggregation of sodium hypochlorite can be inhibited. The reason is that, since each of the ultrafine bubbles has a negative potential and a repulsive force arises between the respective ultrafine bubbles, the sodium hypochlorite can be dispersed, and since the lifetime of the ultrafine bubbles is long, the dispersed state can be maintained.

With respect to the cleaning applications, for example, use as a cleaning fluid for industrial products is expected. In particular, use of the ultrafine bubble water for usages in which microbubbles cannot be used because the bubble size is large (for example, cleaning of state-of-the-art semiconductor substrates) is expected. Because ultrafine bubbles have a negative potential, they can be used to adsorb an oil content or the like that is positively charged. Therefore, a cleaning effect such as removing the oil content can be obtained even without using a surface-active agent or the like.

With respect to the marine product applications, for example, use as water for a water tank or the like for raising hatchery fish is expected. For example, growth promotion that is achieved by reducing the stress of hatchery fish by an effect that eliminates the problem of insufficient oxygen in the water by means of oxygen bubbles can be expected. Further, an effect of preventing decay during storage of fresh fish or sliced fish is achieved by reducing the dissolved oxygen in water by means of nitrogen bubbles, and thus the time period for which freshness is maintained is lengthened. In addition, because live fish can be transported in a state of suspended animation under the influence of an anesthetic effect produced by CO₂ bubbles, there is potential for use of the ultrafine bubble water for increasing the transport quantity and transportation distance of live fish. If microbubbles which have a short lifetime are used, it is necessary to constantly supply microbubbles. Therefore, for example, in a case of transporting live fish or the like, it is necessary to mount a device that generates microbubbles on a transportation vehicle. In contrast, because the lifetime of ultrafine bubbles is long, it suffices to merely generate ultrafine bubble water containing ultrafine bubbles, at the beginning. That is, in the case of transporting live fish or the like, it is not necessary to mount the device that generates ultrafine bubbles on the transportation vehicle

### Other Embodiments

Although the fine bubble generation promoter, the fine-bubble-containing liquid, the device and the method for producing the fine-bubble-containing liquid according to the aspects of the present invention have been described based on the above embodiments, the present invention is not limited to the embodiments.

The constituent elements shown in the accompanying Drawing and/or described in the Detailed Description may include not only constituent elements necessary to solve the previously-described problems but also constituent elements unnecessary to solve the problem in order to explain an example of the above-described technique. Therefore, even if such an unnecessary constituent element is shown in the accompanying Drawing and/or described in the Detailed Description, the constituent element should not be directly understood as necessary one.

The prevent invention also includes various modifications made by those skilled in the art to the embodiments and any combinations of the constituent elements and functions according to the embodiments without departing from the scope of the present invention.

In the foregoing embodiments, the phrase “containing a fatty acid or a fat-soluble vitamin” means containing at least one of the fatty acid and the fat-soluble vitamin. For example, the phrase “containing a fatty acid or a fat-soluble vitamin, and a hydrocarbon” means that both the fatty acid and the fat-soluble vitamin as well as the hydrocarbon may be contained. For example, oleic acid, α-tocopherol, and heptane may be added to the water. Note that, the phrase “concentration of a fatty acid or a fat-soluble vitamin” means a total concentration of the fatty acid and the fat-soluble vitamin in a case where both the fatty acid and the fat-soluble vitamin are contained.

It is thereby possible to produce the same effects as described previously.

Further, although the above description adopts a case in which the fine bubbles are formed of air as an example, the present invention is not limited thereto. For example, the fine bubbles may be formed of oxygen, nitrogen, fluorine, ozone gas, or the like. It suffices to appropriately select the kind of the gaseous matter to be used according to the intended use or the like. For example, by using oxygen as the gaseous matter, the fine-bubble-containing liquid will be effective for promoting growth of crops, marine products, or the like, and by using nitrogen as the gaseous matter, the fine-bubble-containing liquid will be effective for sterilization treatments (maintaining the freshness of food or the like).

### INDUSTRIAL APPLICABILITY

The fine bubble generation promoter, the fine-bubble-containing liquid, and the method and device for producing a fine-bubble-containing liquid according to the present invention are useful for various industrial uses such as cleaning of industrial products including semiconductor devices, growth promotion of crops and marine products, sterilization treatments, and improvement of water quality and soil.

### REFERENCE MARKS IN THE DRAWINGS

1, 1a fine-bubble-containing liquid
2, 2a liquid (ultrapure water)
3, 3a additive agent
4, 4a fine bubble
10 device for producing a fine-bubble-containing liquid
11 pump
12a first piping
12b second piping
13 nozzle (fine bubble generator)
13a inflow portion
13b nozzle pipe
14 water supply valve
15 discharge valve
16 sampling valve
17 injection valve
18 hermetically sealed flow channel

1. (canceled)

2. A fine bubble generation promoter, comprising:
one of a fatty acid and a fat-soluble vitamin, the one of the fatty acid and the fat-soluble vitamin being in an amount within a range of 2.4 wt % to 33 wt %, inclusive; and
a hydrocarbon in an amount within a range of 67 wt % to 97 wt %, inclusive,
wherein a total concentration of (i) the one of the fatty acid and the fat-soluble vitamin and (ii) the hydrocarbon is 99 wt % or more.

3. A fine-bubble-containing liquid, comprising:
water;
one of a fatty acid and a fat-soluble vitamin, the fatty acid comprising only carbon, oxygen, and hydrogen
hydrocarbon; and
fine bubbles,
wherein the fine bubbles each have a size within a range of 1 nm to 800 nm, inclusive,
a concentration of the one of the fatty acid and the fat-soluble vitamin is within a range of 10 ppm to 50 ppm, inclusive, and
a concentration of the hydrocarbon is within a range of 100 ppm to 400 ppm, inclusive.

4. (canceled)

5. A fine-bubble-containing liquid, comprising:
water;
the fine bubble generation promoter according to claim 2; and
fine bubbles,
wherein the fine bubbles each have a size within a range of 1 nm to 800 nm, inclusive.

6. The fine-bubble-containing liquid according to claim 5,
wherein a concentration of the one of the fatty acid and the fat-soluble vitamin is within a range of 10 ppm to 50 ppm, inclusive, and
a concentration of the hydrocarbon is within a range of 100 ppm to 400 ppm, inclusive.

7. The fine-bubble-containing liquid according to claim 5,
wherein the hydrocarbon is an alkane having a carbon number from 5 to 13, inclusive.

8. The fine-bubble-containing liquid according to claim 7,
wherein the hydrocarbon is any one of hexane, heptane, octane, nonane, and decane.

9. The fine-bubble-containing liquid according to claim 3,
wherein a surface of each of the fine bubbles is covered with at least a single one of the fatty acid or covered with at least a single one of the fat-soluble vitamin.

10. The fine-bubble-containing liquid according to claim 3,
wherein the fatty acid is a saturated fatty acid having a carbon number from 5 to 12, inclusive, or is an unsaturated fatty acid having a carbon number of 12 or more, and
the fat-soluble vitamin has a carbon number of 12 or more.

11. The fine-bubble-containing liquid according to claim 10,
wherein the fatty acid is one of an oleic acid, a caprylic acid, a nonanoic acid, a palmitoleic acid, a linoleic acid, an α-linolenic acid, and an arachidonic acid, and
the fat-soluble vitamin is α-tocopherol.

12. The fine-bubble-containing liquid according to claim 3,
wherein the fine bubbles each have a size of not more than 200 nm.

13. The fine-bubble-containing liquid according to claim 3,
wherein a concentration of the fine bubbles is 1×10⁹ per ml or more.

14. The fine-bubble-containing liquid according to claim 3,
wherein an electric resistivity is 1 MΩ·cm or more.

15. The fine-bubble-containing liquid according to claim 3,
wherein a lifetime of the fine bubbles is 30 days or more.

16. A method for producing a fine-bubble-containing liquid, the method comprising:
generating a fine bubble generation promoting liquid by adding one of a fatty acid and a fat-soluble vitamin to water, the fatty acid comprising only carbon, oxygen, and hydrogen; and
generating fine bubbles each having a size within a range of 1 nm to 800 nm, inclusive, in the fine bubble generation promoting liquid, without introducing a gaseous matter from outside.

17. The method according to claim 16,
wherein the generating of the fine bubbles includes jetting out the fine bubble generation promoting liquid from a nozzle in a hermetically sealed flow channel.

18. The method according to claim 16,
wherein in the generating of the fine bubble generation promoting liquid, a hydrocarbon is further added to the water.

19. The method according to claim 18,
wherein the generating of the fine bubble generation promoting liquid includes:
supplying the water into the hermetically sealed flow channel to cause the water to circulate and, discharge a part of the water; and
adding (i) the one of the fatty acid and the fat-soluble vitamin, and (ii) the hydrocarbon into the hermetically sealed flow channel until (i) the one of the fatty acid and the fat-soluble vitamin and (ii) the hydrocarbon reaches a predetermined amount.

20. The method according to claim 19,
wherein in the adding into the heiinetically sealed flow channel, (i) the one of the fatty acid and the fat-soluble vitamin, and (ii) the hydrocarbon are added into the hermetically sealed flow channel until a concentration of the one of the fatty acid and the fat-soluble vitamin reaches a concentration within a range of 10 ppm to 50 ppm, inclusive, and a concentration of the hydrocarbon reaches a concentration within a range of 100 ppm to 400 ppm, inclusive.

21. The method according to claim 16,
wherein the water is ultrapure water with an electric resistivity of 18 MΩ·cm or more.

22. A device for producing a fine-bubble-containing liquid, the device comprising:
a water supply valve that supplies water;
piping that is a channel in which the water flows;
a pump that pumps the water;
an injection valve that injects one of a fatty acid or a fat-soluble vitamin into the water, the fatty acid comprising only carbon, oxygen, and hydrogen; and
a fine bubble generator that generates a fine-bubble-containing liquid having fine bubbles from the water and a fine bubble generation promoting liquid comprising the one of the fatty acid and the fat-soluble vitamin,
wherein the device for producing a fine-bubble-containing liquid does not comprise an introduction valve for introducing a gaseous matter into the water from outside.

23. (canceled)
